(19) United States
(12) Patent Application Publication
(10) Pub. No.: US 2026/0366077 A1
(43) Pub. Date: Nov. 19, 2026

(54) PHOTO-SWITCHABLE SURFACES

(71) Applicant: ILLUMINA, INC., San Diego, CA (US)

(72) Inventors: Weixian XI, San Diego, CA (US); Hua WANG, San Diego, CA (US)

(73) Assignee: ILLUMINA, INC., San Diego, CA (US)

(21) Appl. No.: 19/487,019

(22) PCT Filed: Mar. 24, 2025
(86) PCT No.: PCT/US2025/021183

§ 371 Date: Nov. 24, 2025

Related U.S. Application Data
(60) Provisional application No. 63/570,151, filed on Mar. 26, 2024.

(51) Int. Cl. C12Q 1/6874; B01J 19/00; C12Q 1/6876

(52) U.S. Cl. CPC C12Q 1/6874; B01J 19/0046; C12Q 1/6876; B01J 2219/00608; B01J 2219/0061; B01J 2219/00619; B01J 2219/00623; B01J 2219/00637; B01J 2219/00722

(57) ABSTRACT

This disclosure provides photo-switchable surfaces, e.g., for flow cells, that change in wettability from hydrophobic to hydrophilic when irradiated with a specific wavelength of electromagnetic radiation.

### CROSS-REFERENCE TO RELATED APPLICATIONS

This application is a U.S. National Phase filing under 35 U.S.C. § 371 of International Application No. PCT/US2025/021183, filed Mar. 24, 2025, and claims benefit of priority from U.S. Provisional Patent Application No. 63/570,151, filed Mar. 26, 2024, the entire contents of which applications are hereby incorporated herein by reference in their entireties for all purposes.

### SEQUENCE LISTING

The instant application contains an electronic sequence listing. The contents of the electronic sequence listing IP-2716-US_Sequence_Listing_5317.390A.xml; Size: 3,871 bytes; and Date of Creation: Nov. 21, 2025, is herein incorporated by reference in its entirety.

### TECHNICAL FIELD

The present disclosure provides surfaces, e.g., of flow cells that undergo property changes when exposed to an external stimulus.

### BACKGROUND

Biological arrays are among a wide range of tools used to detect and analyze molecules, including deoxyribonucleic acid (DNA) and ribonucleic acid (RNA). In these applications, the arrays are engineered to include probes for nucleotide sequences present in genes of humans and other organisms. In certain applications, for example, individual DNA and RNA probes are attached to a support, e.g., in a geometric or random grid or array. A test sample, e.g., from a known person or organism is exposed to the probes in the array, such that complementary fragments hybridize to the probes.

Biological arrays can be used, for example, for genetic sequencing. In general, genetic sequencing involves determining the order of nucleotides or nucleic acids in a length of genetic material, such as a fragment of DNA or RNA. Increasingly longer sequences are being analyzed, and the resulting sequence information can be used in various bioinformatics methods to logically fit fragments together so as to determine the sequence of genetic material from which the fragments were derived. Automated, computer-based examination of characteristic fragments have been developed, and have been used in genome mapping, identification of genes and their function, evaluation of risks of certain conditions and disease states, and so forth. Beyond these applications, biological arrays can be used for the detection and evaluation of a wide range of molecules, families of molecules, genetic expression levels, single nucleotide polymorphisms, and genotyping.

### SUMMARY

The present disclosure is based, inter alia, on the concept that sequencing on flow cells can occur on selective portions of the flow cell using a photo-switchable coating. In particular, the methods and compositions of the disclosure relate to, for example, photo-switchable surfaces, e.g., for flow cells, that change in wettability from hydrophobic to hydrophilic when irradiated with a specific wavelength of electromagnetic radiation.

In a first aspect, the disclosure provides coated substrates for sequencing polynucleotide molecules, comprising: primer oligonucleotides attached to a surface of a substrate; and a coating on the surface, wherein the coating comprises one or more components that shift from a first conformation to a second conformation when irradiated with electromagnetic radiation in the wavelength range of 280 nm to 700 nm and wherein the first conformation causes the coating to be hydrophobic and the second conformation causes the coating to be hydrophilic.

In some embodiments, the electromagnetic radiation comprises ultra-violet (UV), visible, or infrared (IR) light. In some embodiments, the coating comprises a polymer. In some embodiments, the polymer comprises a polysaccharide, a poly(allylamine hydrochloride), a polyethylene glycol, or a polystyrene, or a combination of any two or more thereof. In some embodiments, the one or more components comprise a naphthopyran, a diarylethene, a viologen, a spiropyran, an azobenzene, or a fulgide, or a combination of any two or more thereof.

In some embodiments, the one or more components comprise:

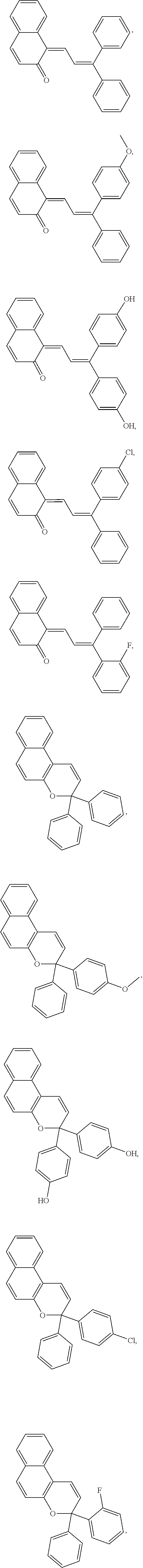

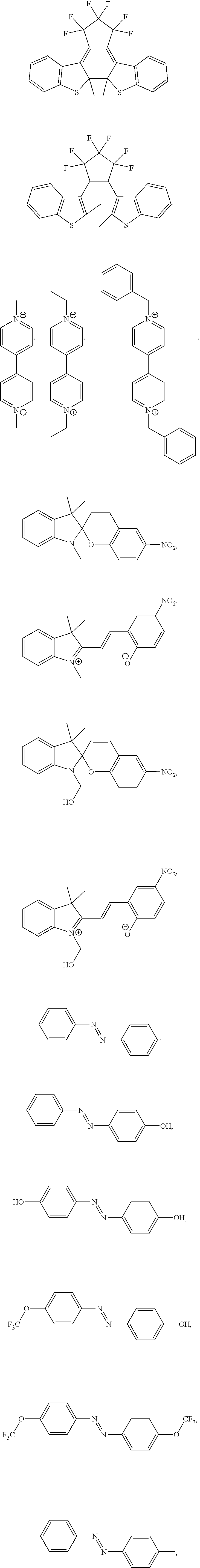

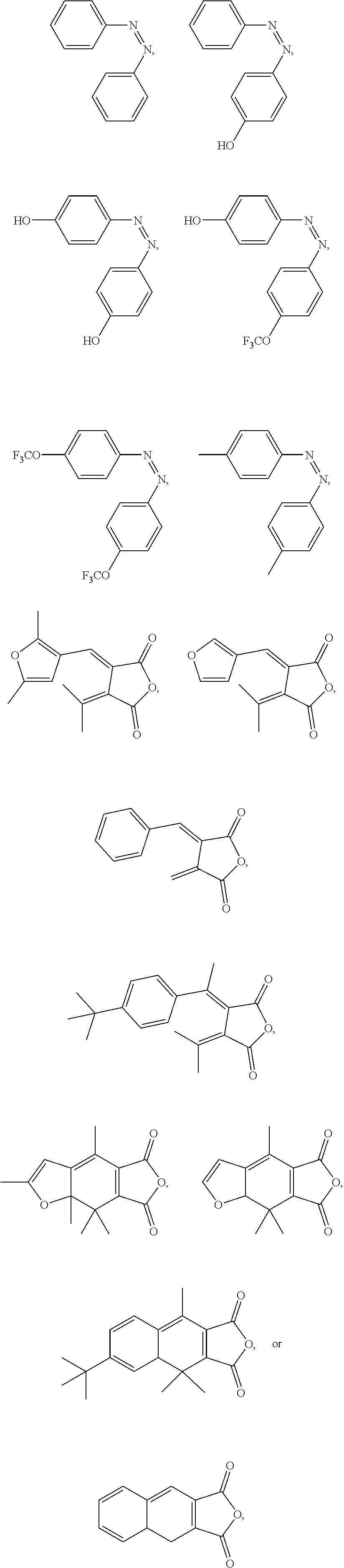

or a configurational isomer thereof, or a salt thereof.

In some embodiments, the contact angle of the coating with the one or more components in the first conformation is great than 90°, great than 120°, or great than 150°. In some embodiments, the contact angle of the coating with the one or more components in the second conformation is less than 90°, less than 30°, or less than 5°. In some embodiments, the one or more components shifts from a first conformation to a second conformation when illuminated with UV light. In some embodiments, the thickness of the coating ranges from about 1 μm to about 15 μm. In some embodiments, the coated substrate for sequencing polynucleotide molecules is part of a segmented random access flow cell. In some embodiments, the coating is present on a portion of the surface. In some embodiments, the coating is present on the entirety of the surface. In some embodiments, the coated substrate further includes a second coating wherein the second coating includes an additional component that shifts from a first conformation to a second conformation when exposed to an external stimulus. In some embodiments, the primer oligonucleotides comprise TTTTTTTTTTAATGATACGGCGACCACCGAGAUCTACAC (SEQ ID NO: 1) or TTTTTTTTTTCAAGCAGAAGACGGCATACGA (8-oxo G) AT (SEQ ID NO: 2).

In another aspect, the disclosure provides methods of preparing a coated substrate for sequencing polynucleotide molecules, the method comprising: obtaining a substrate comprising a surface; attaching a plurality of primer oligonucleotides to one or more areas of the surface by covalent bonding or non-covalent bonding to provide an oligonucleotide bound surface; and applying a photo-switchable polymer to the oligonucleotide bound surface forming a polymer coating to provide the coated substrate for sequencing.

In some embodiments, the primer oligonucleotides are immobilized on the surface through hybridization. In some embodiments, applying the photo-switchable polymer to the templated substrate comprises spin coating, precision nozzle dispersion, or drop coating. In some embodiments, the method further comprises curing the photo-switchable polymer. In some embodiments, the method further comprises polishing or planarizing the coated substrate.

In another aspect, the disclosure provides methods of sequencing a polynucleotide, the method comprising: illuminating one or more areas of a substrate coated with a photo-switchable polymer in a hydrophobic, locked state with electromagnetic radiation having a wavelength of about 260 nm to about 380 nm to activate the polymer in the illuminated areas to form hydrophilic, unlocked areas on the substrate; sequencing polynucleotides in the unlocked areas; rinsing the unlocked areas; and illuminating the unlocked areas with electromagnetic radiation having a wavelength of about 380 nm to about 1000 nm to deactivate the photo-switchable polymer, thereby returning the previously unlocked areas to a hydrophobic, locked state.

In some embodiments, the method comprises illuminating the entire area of the substrate coated with the photo-switchable polymer. In some embodiments, the contact angle of the coating with the photo-switchable polymer in a hydrophobic, locked state is great than 90°, great than 120°, or great than 150°. In some embodiments, the contact angle of the coating with the photo-switchable polymer in a hydrophilic, unlocked state is less than 90°, less than 30°, or less than 5°. In some embodiments, illuminating one or more areas of a substrate coated with a photo-switchable polymer in a hydrophobic, locked state is done with electromagnetic radiation having a wavelength of about 300 nm to about 380 nm. In some embodiments, illuminating the unlocked areas are done with electromagnetic radiation having a wavelength of about 380 nm to about 780 nm. In some embodiments, the photo-switchable polymer coating comprises poly(allylamine hydrochloride) modified with 3-(aminopropyl)triethoxysilane and 7-[(trifluoromethoxy-phenylazo)phenoxy]pentanoic acid. In some embodiments, the unlocked areas are illuminate with electromagnetic radiation having a wavelength of about 440 nm. In some embodiments, the sequencing of the polynucleotides comprises sequencing by synthesis. In some embodiments, rinsing the unlocked areas comprises rinsing with water.

In another aspect, the disclosure provides methods of sequencing a polynucleotide, the method comprising: illuminating one or more areas of a coated substrate disclosed herein with electromagnetic radiation having a wavelength of about 260 nm to about 380 nm to activate the coated substrate in the illuminated areas to form hydrophilic, unlocked areas on the substrate; sequencing polynucleotides in the unlocked areas; rinsing the unlocked areas; and illuminating the unlocked areas with electromagnetic radiation having a wavelength of about 380 nm to about 1000 nm to deactivate the photo-switchable polymer, thereby returning the previously unlocked areas to a hydrophobic, locked state.

In some embodiments, illuminating comprising illuminating the entire area of the substrate coated with the photo-switchable polymer. In some embodiments, the contact angle of the coating in a hydrophobic, locked state is great than 90°, great than 120°, or great than 150°. In some embodiments, the contact angle of the coated substrate with a photo-switchable polymer in a hydrophilic, unlocked state is less than 90°, less than 30°, or less than 5°. In some embodiments, illuminating one or more areas of a substrate coated with a photo-switchable polymer in a hydrophobic, locked state is done with electromagnetic radiation having a wavelength of about 300 nm to about 380 nm. In some embodiments, illuminating the unlocked areas is done with electromagnetic radiation having a wavelength of about 380 nm to about 780 nm. In some embodiments, sequencing polynucleotides comprises sequencing by synthesis. In some embodiments, rinsing the unlocked areas comprises rinsing with water.

As used herein a “hydrophobic surface” refers to a surface with a static water contact angle of greater than or equal to 90°. A “superhydrophobic surface” refers to a surface with a static water contact angle of greater than 150°.

As used herein a “hydrophilic surface” refers to a surface with a static water contact angle of less than 90°. As used herein a “superhydrophilic surface” refers to a surface with a static water contact angle of less than 5°.

As used herein the terms “polynucleotide” and “oligonucleotide” are used interchangeably to refer to nucleic acid molecules of any length, and can comprise ribonucleotides, deoxyribonucleotides, analogs thereof, or mixtures thereof. This term refers only to the primary structure of the nucleic acid molecules. Thus, the term includes triple-, double-, and single-stranded deoxyribonucleic acid (“DNA”), as well as triple-, double-, and single-stranded ribonucleic acid (“RNA”).

As used herein, “primer oligonucleotide” or “primer” are defined as a single stranded nucleic acid sequence (e.g., single strand DNA or single strand RNA). Some primers, serve as a starting point for template amplification and cluster generation. Other primers, serve as a starting point for DNA or RNA synthesis. The 5′ terminus of the primer can be modified to allow a coupling reaction with a surface. The primer length can be any number of bases long and can include a variety of non-natural nucleotides. In an example, the sequencing primer is a short strand, ranging from 10 to 60 bases, or from 20 to 40 bases.

As used herein, “target sequences” and its derivatives, refers generally to a nucleic acid sequence produced by making copies of the sequences of interest using primers. The target sequences can be either of the same sense (e.g., the positive strand) or antisense (i.e., the negative strand) with respect to the sequence of interest.

As used herein the terms “polynucleotide template” and “template polynucleotide” both refer to a nucleic acid molecule that includes a target nucleic acid molecule and an adaptor on one or both ends.

Suitable nucleotides for use in the methods disclosed herein include, but are not limited to, deoxynucleotide triphosphates, deoxyadenosine triphosphate (dATP), deoxythymidine triphosphate (dTTP), deoxycytidine triphosphate (dCTP), and deoxyguanosine triphosphate (dGTP). Optionally, the nucleotides used in the methods disclosed herein, whether labeled or unlabeled, can include a blocking moiety such as a reversible terminator moiety that inhibits chain extension. Suitable labels for use on the labeled nucleotides include, but are not limited to, haptens, radionucleotides, enzymes, fluorescent labels, chemiluminescent labels, and chromogenic agents.

As used herein, the term “alkyl” refers to a straight or branched hydrocarbon chain that is fully saturated (i.e., contains no double or triple bonds). The alkyl group can have 1 to 20 carbon atoms. Example alkyl groups include methyl, ethyl, propyl, isopropyl, butyl, isobutyl, tertiary butyl, pentyl, hexyl, and the like. As an example, the designation “C₁₋₆ alkyl” indicates that there are one to six carbon atoms in the alkyl chain, for example, the alkyl chain is selected from the group consisting of methyl, ethyl, propyl, butyl, pentyl, hexyl, iso-propyl, sec-butyl, and t-butyl.

As used herein, the term “flow cell” refers to a vessel having a chamber (i.e., flow channel) where a reaction can be carried out, an inlet for delivering reagent(s) to the chamber, and an outlet for removing reagent(s) from the chamber. In some embodiments, the chamber enables the detection of the reaction that occurs in the chamber. For example, the chamber can include one or more transparent surfaces allowing for the optical detection of arrays, optically labeled molecules, or the like, in the chamber. In some embodiments, the flow cell is a 10 cm-25 mm×75 mm standard HiSeq® flow cell with 8 lanes. The flow cell can be a random access flowcell, which is designed to allow for selective sequencing of individual DNA molecules or regions of interest within a complex sample rather than sequencing the entire sample indiscriminately. A random access flowcell can enhances efficiency by focusing resources on specific areas of interest and significantly reducing the volume of data that needs to be processed and analyzed.

As used herein, the term “YES® method” refers to a chemical vapor deposition process developed by Illumina, Inc. which uses the chemical vapor deposition tool provided by Yield Engineering Systems (“YES®”). The tool includes three different vapor deposition systems. The automated YES®-VertaCoat silane vapor system is designed for volume production with a flexible wafer handling module that can accommodate 200 mm or 300 mm wafers. The manual load YES®-1224P Silane Vapor System is designed for versatile volume production with its configurable large capacity chambers. YES®-LabKote is a low-cost, tabletop version that is ideal for feasibility studies and for R&D.

As used herein, the term “λₘₐₓ” refers to the wavelength of maximum light absorption. As used herein, the term “substrate die” is the either the bottom or top part of the flow cell that is diced from the 200 mm or 300 mm wafer.

Conventional sequencing flow cells generally require full usage of all of the lanes for clustering and sequencing. This requires that an entire flow cell is used even for small samples to avoid cross-contamination. Dedicating an entire lane to one sample is not cost effective and can result in wasted reagents. To avoid this disadvantage, the present disclosure enables small scale samples to be run on separate areas of large flow cells, without the risk of cross-contamination, because the desired areas can be selectively “unlocked,” while the rest of the flow cell remains “locked” and available for a future use. Thus, customers do not need to discard large flow cells where only a small portion of the flow cell was used, and they will not need to pool samples. The present disclosure enables multiple and multiplexed uses of the same flow cell. These advantages save both money and materials for customers. The present disclosure can also be used on a wide variety of flow cells, including closed flow cells and open flow cells.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated herein by reference in their entirety. In case of conflict, the present specification, including definitions, will control.

Other features and advantages of the invention will be apparent from the following detailed description and figures, and from the claims.

### BRIEF DESCRIPTION OF THE DRAWINGS

FIGS. 1A to 1C are a series of schematic diagrams of examples of processes described herein.

FIGS. 2A to 2D are a series of a schematic cross-sectional views depicting examples of different substrates disclosed herein.

FIG. 3 is a schematic of photo-reactive components disclosed herein.

FIG. 4 is a flow diagram illustrating an example of a preparation method disclosed herein.

FIG. 5 is a flow diagram illustrating an example of a sequencing method disclosed herein.

### DETAILED DESCRIPTION

This disclosure provides photo-switchable surfaces, e.g., within or on flow cells and other surfaces, e.g., on or in other devices, with hydrophobic coatings that change in wettability from hydrophobic to hydrophilic when irradiated with specific wavelengths of electromagnetic radiation, such as ultraviolet (UV), infrared (IR), or visible light. Since flow cell reactions use reagents that are mostly water-based, the reactions take place only on the hydrophilic areas of the flow cell. The flow cells and other surfaces described herein can also be stored with the hydrophobic coating to protect against degradation of materials, e.g., polynucleotides bound to the surface of the flow cells, before the hydrophobic coating is applied.

Before a flow cell with the hydrophobic coating, e.g., in a hydrophobic state, is used for a reaction (e.g., clustering or sequencing), the entire flow cell or one or more areas of the flow cell are irradiated with the same wavelength of electromagnetic radiation, causing the irradiated area or areas on the surface to become hydrophilic, e.g., in a hydrophilic state. The hydrophobic area or areas are considered “locked,” and the hydrophilic area or areas are considered “unlocked.” The desired reagents are added to the flow cell, and the corresponding reaction takes place only on the unlocked, hydrophilic areas. Once the reaction is complete, the flow cell can be rinsed and irradiated with light deactivating the hydrophilic areas of the surface and returning the entire surface to the hydrophobic state. This can be repeated on one or more new areas of the flow cell.

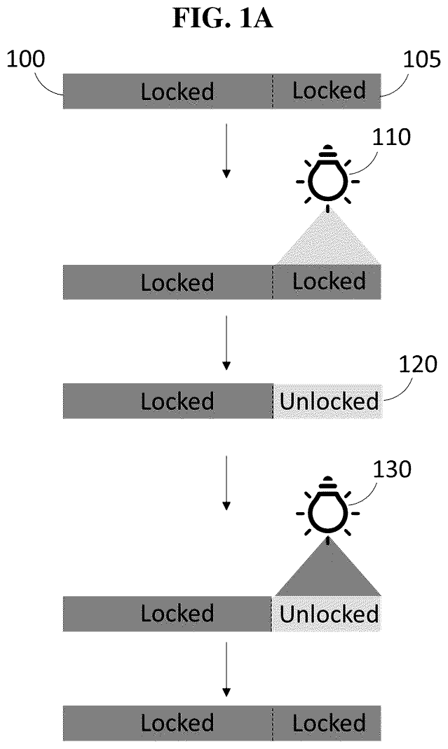

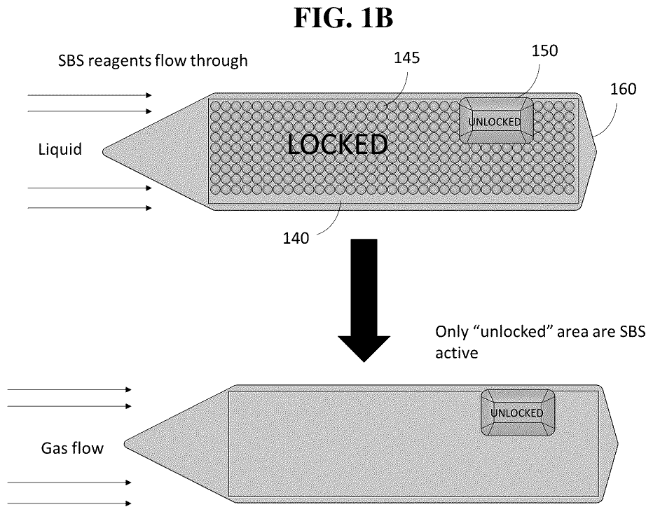

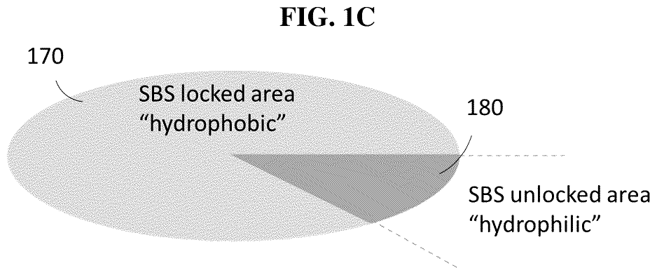

FIG. 1A shows a graphical representation of the processes disclosed herein. A flow cell 100 is in a locked, hydrophobic state. It is then irradiated with light 110, e.g., having a wavelength of about 365 nm, if the coating is made of poly(allylamine hydrochloride) modified with 3-(aminopropyl)triethoxysilane and 7-[(trifluoromethoxyphenylazo)phenoxy]pentanoic acid. The area 105 that is irradiated with light 110 enters the unlocked state 120, and thus becomes hydrophilic. The flow cell with a portion unlocked can be used for sequencing. Once the sequencing reaction is complete, the unlocked area on the flow cell is irradiated with a light 130 of a different wavelength of about 440 nm that returns the unlocked area 105 to the locked state.

FIG. 1B shows a graphical representation of the processes disclosed herein. The flow cell 160 has a locked, hydrophobic region 140 including nanowells 145 and an unlocked, hydrophilic region 150. Sequencing by synthesis (SBS) reagents are added to the flow cell 160 in the form of an aqueous solution. The sequencing reaction only occurs on the unlocked, hydrophilic region 150. Once the SBS reaction is complete, gas (e.g., nitrogen or argon) is applied to the flow cell 160 to blow the aqueous solution.

FIG. 1C shows a graphical representation of a circular flow cell. The locked, hydrophobic region 170 of the flow cell is the region of the flow cell where an SBS reaction cannot take place. In one embodiment, the unlocked, hydrophilic region 180 of the flow cell, which is the region of the flow cell where an SBS reaction can take place, is shown here as a wedge-shaped portion of the circular flow cell.

In some embodiments, there can be two or more different types of hydrophobic coatings in different individual or multiple areas or groups of areas, e.g., in random or specific patterns, on the surface, e.g., that are unlocked using two or more, e.g., 2, 3, 4, 5, 6, or more different wavelengths of electromagnetic radiation, wherein one or more of the different areas can be unlocked using the same wavelength, or a different wavelength for each area or group of areas in selected patterns. For example, surfaces within each of 8 different lanes within a flow cell can be coated with a different hydrophobic coating, or even numbered lanes can each have the same type of coating, and odd numbered lanes can each have a second type of coating. Or individual lanes can each have two or more different coatings in different patterns.

### Coated Surfaces on Substrates

This disclosure provides different coated substrates for sequencing that include primer oligonucleotides (also referred to as primers) attached to a surface and a coating on the surface.

### Substrate

A substrate is made of material that has a rigid or semi-rigid structure and a surface to which a polynucleotide can be attached or upon which nucleic acids can be synthesized and/or modified. Substrates can include, for example, any resin, gel, bead, well, column, chip, flow cell, membrane, matrix, plate, filter, glass, controlled pore glass (CPG), polymer support, membrane, paper, plastic, plastic tube or tablet, plastic bead, glass bead, slide, ceramic, silicon chip, multi-well plate, nylon membrane, fiber optic, and PVDF membrane. In some embodiments, the substrate is glass. In some embodiments, the substrate is resin on glass.

In some embodiments, the substrate is within or a part of a flow cell, e.g., a flow cell can include a substrate comprising an exposed surface. In some embodiments, the flow cell includes a lid bonded to a region of the substrate, wherein the lid and the substrate at least partially define a flow channel. In some embodiments, the flow channel is etched into the substrate. The flow cell can have one or more flow channels. For example, the flow cell can have 1, 2, 3, 4, 5, 6, 7, or 8, or more flow channels. In some embodiments, the flow channels can be separated regions, without any etching, on the surface of the substrate, e.g., a glass substrate. In other embodiments, the flow channels can be cut, etched, or molded into the surface of the substrate.

The patterned surfaces can use nanowells for cluster generation to make more efficient use of the surface area of the flow cell. This flow cell design contributes to increased data output, reduced costs, and faster run times.

Patterned flow cells can contain 100s of thousands, million, or billions to tens of billions of nanowells at fixed locations across both surfaces of the flow cell. The structured organization can provide even spacing of sequencing clusters. Clusters or oligonucleotides can form in the nanowells, making the flow cells less susceptible to overloading and more tolerant to a broader range of library densities. Precise nanowell positioning can eliminate the need to map cluster sites and can save hours on each sequencing run. Higher cluster density can lead to more usable data per flow cell, driving down the cost per gigabase (Gb) of the sequencing run.

In some embodiments, patterned flow cells can be produced using semiconductor manufacturing technology. For example, starting with a glass substrate, patterned nanowells are etched into the surface for optimal cluster spacing. Each nanowell contains DNA probes used to capture prepared DNA strands for amplification during cluster generation. The regions between the nanowells are devoid of DNA probes. The process ensures that DNA clusters only form within the nanowells, providing even, consistent spacing between adjacent clusters and allowing accurate resolution of clusters during imaging. Maximal use of the flow cell surface leads to overall higher clustering.

These methods can increase data output. Exclusion amplification allows simultaneous seeding (landing of the DNA strand in the nanowell) and amplification during cluster generation, which can reduce the chances of multiple library fragments amplifying in a single cluster. This method maximizes the number of nanowells occupied by DNA clusters originating from a single DNA template, increasing the amount of usable data from each run.

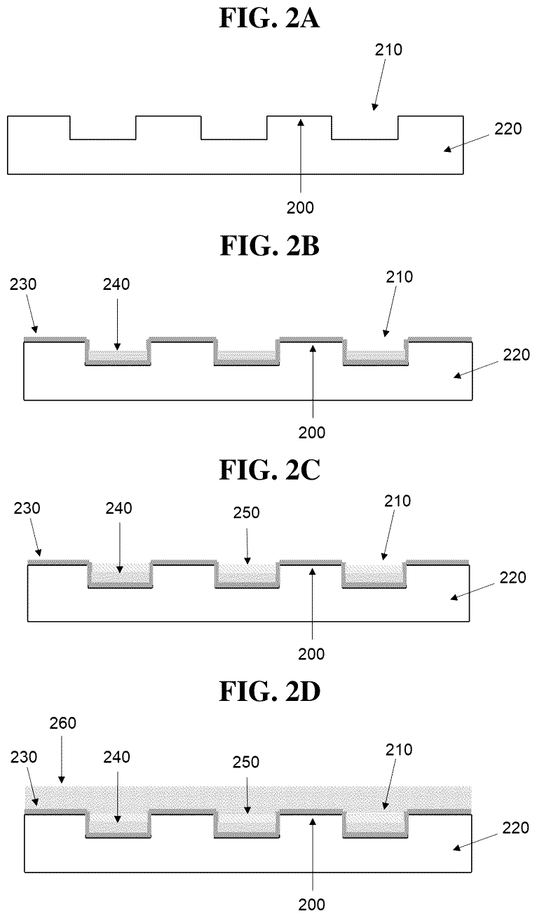

FIG. 2A is a cross-sectional view of an example of the patterned substrate 220. The patterned substrate 220 can be a patterned wafer or a patterned die or any other patterned substrate (e.g., panel, rectangular sheet, etc.). Any type of solid substrate can be used. The patterned substrate can be used to form several flow cells, and a patterned die can be used to form a single flow cell. In an example, the substrate can have a diameter ranging from about 2 mm to about 300 mm, or a rectangular sheet or panel having its largest dimension up to 10 feet (~3 meters). In some embodiments, the substrate wafer can have dimensions ranging from about 200 mm to about 300 mm. In some embodiments, the substrate die has a width ranging from about 0.1 mm to about 10 mm. While examples of dimensions have been provided, it is to be understood that substrates with any suitable dimensions can be used.

In this example, the patterned substrate 220 includes depressions 210 defined on or in an exposed layer or surface of the substrate 220, and interstitial regions 200 separating adjacent depressions 210. In some embodiments, the depressions 210 become functionalized with surface chemistry, while the interstitial regions 200 can be used for bonding but are typically not designed to have primers attached thereon.

The depressions 210 can be fabricated in or on the substrate 220 using a variety of techniques, including, for example, photolithography, nanoimprint lithography, stamping techniques, embossing techniques, molding techniques, microetching techniques, printing techniques, etc. The technique used will depend on the composition and shape of the substrate 220.

Many different layouts of the depressions 210 can be envisaged, including regular, repeating, and non-regular patterns. In some embodiments, the depressions 210 are disposed in a hexagonal grid for close packing and improved density. Other layouts can include, for example, rectilinear (i.e., rectangular) layouts, triangular layouts, and so forth. In some examples, the layout or pattern can be an x-y format of depressions 210 that are in rows and columns. In some other examples, the layout or pattern can be a repeating arrangement of depressions 210 and/or interstitial regions 200. In still other examples, the layout or pattern can be a random arrangement of depressions 210 and/or interstitial regions 200. The pattern can include spots, pads, wells, posts, stripes, swirls, lines, triangles, rectangles, circles, arcs, checks, plaids, diagonals, arrows, squares, and/or cross-hatches.

The layout or pattern can be characterized with respect to the density of the depressions 210 (i.e., number of depressions 210) in a defined area. For example, the depressions 210 can be present at a density of approximately 2 million per mm². The density can be tuned to different densities including, for example, a density of at least about 100 per mm², about 1,000 per mm², about 0.1 million per mm², about 1 million per mm², about 2 million per mm², about 5 million per mm², about 10 million per mm², about 50 million per mm², or more. Alternatively, the density can be tuned to be no more than about 50 million per mm², about 10 million per mm², about 5 million per mm², about 2 million per mm², about 1 million per mm², about 0.1 million per mm², about 1,000 per mm², about 100 per mm², or less. It is to be further understood that the density of depressions 210 on the substrate 220 can be between one of the lower values and one of the upper values selected from the ranges above. As examples, a high density array can be characterized as having depressions 210 separated by less than about 100 nm, a medium density array can be characterized as having depressions 210 separated by about 400 nm to about 1 μm, and a low density array can be characterized as having depressions 210 separated by greater than about 1 μm. While example densities have been provided, it is to be understood that substrates with any suitable densities can be used.

The layout or pattern can also or alternatively be characterized in terms of the average pitch, i.e., the spacing from the center of the depression 210 to the center of an adjacent interstitial region 200 (center-to-center spacing). The pattern can be regular, such that the coefficient of variation around the average pitch is small, or the pattern can be non-regular in which case the coefficient of variation can be relatively large. In either case, the average pitch can be, for example, at least about 10 nm, about 0.1 μm, about 0.5 μm, about 1 μm, about 5 μm, about 10 μm, about 100 μm, or more. Alternatively, the average pitch can be, for example, at most about 100 μm, about 10 μm, about 5 μm, about 1 μm, about 0.5 μm, about 0.1 μm, or less. In some embodiments, the average pitch is about 200 nm to 700 nm. The average pitch for a particular pattern of sites 200 can be between one of the lower values and one of the upper values selected from the ranges above. In an example, the depressions 210 have a pitch (center-to-center spacing) of about 1.5 μm. While example average pitch values have been provided, it is to be understood that other average pitch values can be used.

In some embodiments, the depressions 210 are wells, and thus the patterned substrate 220 includes an array of wells 210 in a surface thereof. The wells 210 can be microwells or nanowells. The size of each well 210 can be characterized by its volume, well opening area, depth, and/or diameter. The minimum or maximum volume can be selected, for example, to accommodate the throughput (e.g., multiplexity), resolution, analyte composition, or analyte reactivity expected for downstream uses of the flow cell. For example, the volume can be at least about 1×10⁻³ μm³, about 1×10⁻² μm³, about 0.1 μm³, about 1 μm³, about 10 μm³, about 100 μm³, or more. Alternatively, the volume can be at most about 1×10⁴ μm³, about 1×10³ μm³, about 100 μm³, about 10 μm³, about 1 μm³, about 0.1 μm³, or less. It is to be understood that the functionalized coating layer can fill all or part of the volume of a well 210. The volume of the coating layer in an individual well 210 can be greater than, less than or between the values specified above.

The area occupied by each well opening (area) on a surface can be selected based upon similar criteria as those set forth above for well volume. For example, the area for each well opening on a surface can be at least about 1×10⁻³ μm², about 1×10⁻² μm², about 0.1 μm², about 1 μm², about 10 μm², about 100 μm², or more. Alternatively, the area can be at most about 1×10³ μm², about 100 μm², about 10 μm², about 1 μm², about 0.1 μm², about 1×10⁻² μm², or less. The area occupied by each well opening can be greater than, less than or between the values specified above.

The depth of each well 210 can be at least about 0.1 μm, about 1 μm, about 10 μm, about 100 μm, or more. Alternatively, the depth can be at most about 1×10³ μm, about 100 μm, about 10 μm, about 1 μm, about 0.1 μm, or less. The depth of each well 210 can be greater than, less than or between the values specified above.

In some embodiments, the diameter of each well 210 can be at least about 50 nm, about 0.1 μm, about 0.5 μm, about 1 μm, about 10 μm, about 100 μm, or more. Alternatively, the diameter can be at most about 1×10³ μm, about 100 μm, about 10 μm, about 1 μm, about 0.5 μm, about 0.1 μm, or less (e.g., about 50 nm). The diameter of each well 210 can be greater than, less than or between the values specified above.

FIG. 2B is a cross-sectional view of another example of the patterned substrate 220. To obtain the patterned substrate illustrated in FIG. 2B, the patterned substrate shown in FIG. 2A has a silanization layer 230 applied across the surface of patterned substrate 220 including the depressions 210 (e.g., on the bottom surface and along the side walls) and interstitial regions 200. The silanization layer 230 can comprise (3-aminopropyl)triethoxysilane (APTES) or (3-aminopropyl)trimethoxysilane (APTMS) or derivatives thereof. In addition, a functionalized polymer layer 240 can be grafted onto the silanization layer 230, e.g., in the depressions 210. The functionalized polymer layer 240 can include poly(N-(5-azidoacetamidylpentyl)acrylamide)-co-acrylamide (PAZAM), or any other molecule that is functionalized to interact with the patterned substrate 220 and the subsequently applied primer oligonucleotides.

### Primer Oligonucleotides

The substrate can include a population of primer oligonucleotides that are immobilized on the surface. For example, the primer oligonucleotides can be covalently attached to the surface. FIG. 2C shows an example of a patterned substrate 220 with primer oligonucleotides 250 grafted to the functionalized polymer layer 240 in depressions 210.

The primer oligonucleotides are generally configured to bind or hybridize to a portion of a polynucleotide template from a target polynucleotide to be sequenced, e.g., to a portion of an adapter of the polynucleotide template. For example, there can be two or more different types of primer oligonucleotides. In general, the primer oligonucleotides are attached to the surface at the 5′ end and have a free 3′ end. The population of primer oligonucleotides can include a population of a first primer oligonucleotide and a population of a second primer oligonucleotide where the first primer oligonucleotide and the second primer oligonucleotide have different sequences. In some embodiments, additional populations of primer oligonucleotides having sequences different from the first and second primer oligonucleotides are present. The primer oligonucleotides can include un-cleavable primers and/or cleavable primers. The un-cleavable primers and the cleavable primers can be oligo pairs where the un-cleavable primer is a forward amplification primer, and the cleavable primer is a reverse amplification primer. The cleavable primers include a cleavage site, while the un-cleavable primers do not include a cleavage site. In some embodiments, the un-cleavable primer and the cleavable primer have the same nucleotide sequence except that the cleavable primer includes a cleavage site integrated into the nucleotide sequence or a linker attached to the nucleotide sequence. Examples of suitable cleavage sites include enzymatically cleavable nucleobases or chemically cleavable nucleobases, modified nucleobases, or linkers (e.g., between nucleobases). The enzymatically cleavable nucleobase can be susceptible to cleavage by reaction with a glycosylase and an endonuclease, or with an exonuclease. One specific example of the cleavable nucleobase is deoxyuracil (dU), which can be targeted by the Uracil-Specific Excision Reagent (USER) enzyme. Other basic sites can also be used. Examples of the chemically cleavable nucleobases, modified nucleobases, or linkers include a vicinal diol, a disulfide, a silane, an azobenzene, a photocleavable group, allyl T (a thymine nucleotide analog having an allyl functionality), allyl ethers, or an azido functional ether.

In some embodiments, the primer oligonucleotides comprise TTTTTTTTTTAATGATACGGCGACCACCGAGAUCTACAC (SEQ ID NO: 1) or TTTTTTTTTTCAAGCAGAAGACGGCATACGA (8-oxo G) AT (SEQ ID NO: 2).

### Photo-Switchable Coatings

As shown in FIG. 2D, a photo-switchable coating 260 is applied to the patterned substrate 220. As above, the primer oligonucleotides 250 are grafted to the functionalized polymer layer 240 in depressions 210, and the photo-switchable coating 260 is formed on top of the layers 240 and 250, and on at least a portion of the patterned flow cell substrate 220. In some embodiments, the photo-switchable coating 260 is deposited on the interstitial regions 200 and on the depressions 210. In some embodiments, the photo-switchable coating 260 is deposited on the depressions 210 and not the interstitial regions 200.

In some embodiments, the photo-switchable coating is insoluble in water. In some embodiments, the photo-switchable coating includes a polymer. In some embodiments, the photo-switchable coating includes a polyacrylamide, poly(acrylic acid) or polyacrylate (e.g., sodium polyacrylate), poly(methacrylic acid), poly(vinyl pyrrolidone), poly(vinyl alcohol), poly (methacrylamide), a poly(N-alkyl acrylamide), a poly(N-dialkyl acrylamide), poly(N-(2-hydroxypropyl) methacrylamide), poly(divinyl ether-maleic anhydride), a poly(phosphate), a poly(2-alkyl-2-oxazoline), poly(hydroxyethyl methacrylate), poly(2-hydroxyethyl acrylate), polyethylene glycol, poly(sulfobetaine methacrylate), a polyether (e.g., a polyvinyl ether, polyethylene glycol, poly(ethylene oxide), and the like), poly(vinyl ether-maleic acid), a hydroxyl functional polymer (e.g., PEG or PVA), a non-natural polypeptide (e.g., poly(glutamic acid) or a salt thereof), or a silicone, or a combination thereof; including, for example, random, block, and graft copolymers and branched analogs. In some embodiments, the photo-switchable coating includes a polyvinyl alcohol/polyethylene glycol graft copolymer. In some embodiments, photo-switchable coating includes about 75% polyvinyl alcohol and about 25% polyethylene glycol. In some embodiments, the photo-switchable coating includes a poly(N-(5-azidoacetamidylpentyl) acrylamide)-co-acrylamide (PAZAM).

Other examples of suitable materials suitable for use as a photo-switchable coating include polymers having a colloidal structure, such as agarose; or a polymer mesh structure, such as gelatin; or a cross-linked polymer structure, such as polyacrylamide polymers and copolymers, silane free acrylamide (SFA, see, for example, U.S. Patent Publication No. 2011/0059865), or an azidolyzed version of SFA. Examples of suitable polyacrylamide polymers can be formed from acrylamide and an acrylic acid or an acrylic acid containing a vinyl group as described, for example, in WO 2000/031148 or from monomers that form [2+2] photo-cycloaddition reactions, for example, as described in WO 2001/001143 or WO 2003/0014392. Other suitable polymers are co-polymers of SFA and SFA derivatized with a bromo-acetamide group (e.g., N-(5-bromoacetamidylpentyl) acrylamide), or co-polymers of SFA and SFA derivatized with an azido-acetamide group.

In some embodiments, the polymer includes a polysaccharide, a poly(allylamine hydrochloride), a polyethylene glycol, or a polystyrene, or a combination of any two or more thereof.

The photo-switchable coating can protect the primer oligonucleotides during subsequent processing techniques (e.g., assembly techniques, such as lid bonding, wafer dicing, etc.) and/or during shipping of the flow cell and/or during short and/or long-term storage of the flow cell. The storage period can range from any time after the coating has been applied until it is desirable to use the flow cell. In some embodiment, the storage period can be up to 120 days, or longer. In some embodiments, the storage period can range from about 1 day to about 75 days. In some embodiments, the storage period can range from about 1 day to about 2 years, or can be about 6 months, 12 months, 18 months, or 24 months. The photo-switchable coating can protect the primer oligonucleotides from chemical or physical degradation, e.g., from debris and/or contamination that can otherwise contact the surface chemistry during lid bonding or other assembly processes.

For example, the photo-switchable coating can protect the surface chemistry from scratches or other handling related defects that can result during shipping. The photo-switchable coating protects the surface chemistry from environmental factors (e.g., temperature, humidity, etc.) during manufacturing, shipping, and/or short and/or long-term storage (e.g., at a temperature ranging from about 4 °C to about 80 °C, or in some instances lower temperatures, down to about −25 °C). In addition, the photo-switchable coating helps to maintain the stability of the surface chemistry, and thus improves the shelf life, temperature tolerance, durability, and ambient storage capability of the flow cell. The stabilization of the surface chemistry is an efficient process, and the surface chemistry is then stable over time.

In some embodiments, the thickness or depth of the photo-switchable coating is at least about 25 nm. In some embodiments, the thickness or depth of the coating ranges from about 50 nm to about 100 nm. In some embodiments, the thickness or depth of the coating ranges from about 1 μm to about 15 μm. In some embodiments, the thickness or depth of the coating ranges from about 1.5 μm to about 12 μm. The upper limit on the thickness can depend, at least in part, upon the architecture and dimensions of the flow channel, depressions, e.g., nanowells, in the surface of the substrate, and the flow cell that is formed. In an example, the upper end of the thickness range can range from about 10 μm to about 15 μm.

### Coating Components

The coatings described herein also include one or more photo-reactive components in addition to the polymeric material. The coatings can, for example, include one, two, three, four, or five different types of photo-reactive components. In some embodiments, the coating includes one photo-reactive component. In some embodiments, the coating includes two different types of photo-reactive components. The coating can include more than one type of photo-reactive component and the photo-reactive components are evenly dispersed over the surface. In some embodiments, the coating includes more than one photo-reactive component, and the different photo-reactive components are on different areas of the surface.

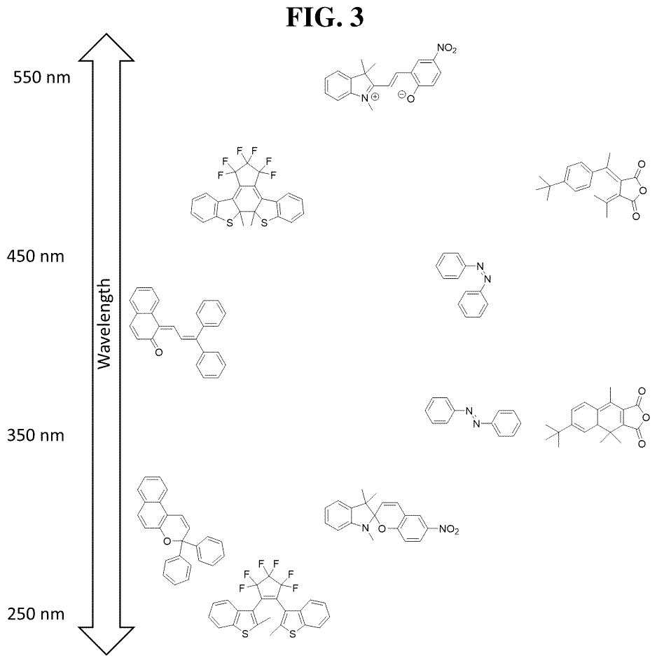

In some embodiments, the one or more photo-reactive components include a naphthopyran, a diarylethene, a viologen, a spiropyran, an azobenzene, or a fulgide, or combination thereof. In some embodiments, the one or more photo-reactive components is a naphthopyran, a diarylethene, a viologen, a spiropyran, an azobenzene, or a fulgide, or combination thereof. Examples of photo-reactive components are shown in FIG. 3. The compounds in FIG. 3 are generally positioned (from top to bottom) according to their maximum UV/Vis absorbance.

In some embodiments, the one or more photo-reactive components include a naphthopyran. In some embodiments, the naphthopyran is a compound of Formula Ia, Formula Ib, or a configurational isomer thereof:

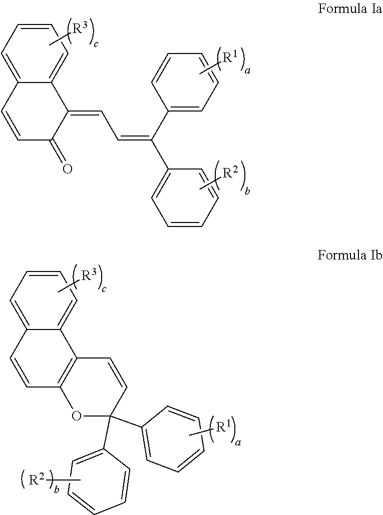

wherein
R¹, R², and R³ are each independently —C₁₋₆ alkyl, —NH₂, —NHCH₃, —N(CH₃)₂, —OH, —OCH₃, —OCH(CH₃)₂, —OCF₃, —CF₃, —Cl, —Br, or —F; and
a, b, and c are each independently 0, 1, 2, or 3.

In some embodiments, the compound of Formula Ia has a λₘₐₓ of about 400 nm to about 530 nm. In some embodiments, the compound of Formula Ia has a λₘₐₓ of about 430 nm. In some embodiments, the compound of Formula Ib has a λₘₐₓ of about 300 nm to about 380 nm. In some embodiments, the compound of Formula Ib has a λₘₐₓ of about 305 nm to about 320 nm.

In some embodiments, the naphthopyran, or configurational isomer thereof, is

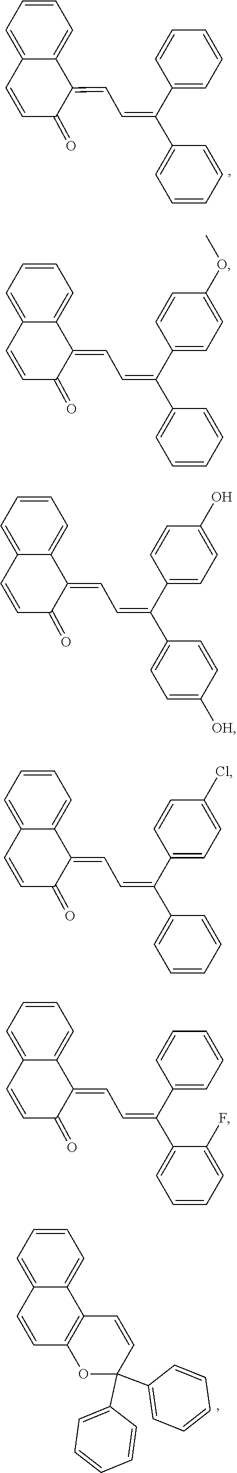

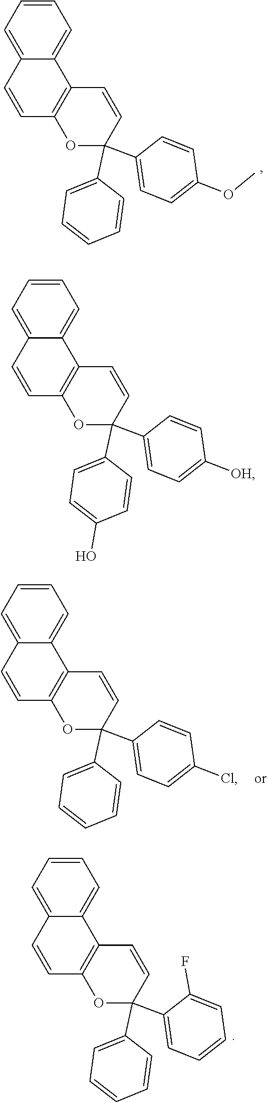

In some embodiments, the one or more photo-reactive components include a diarylethene. In some embodiments, the diarylethene is a compound of Formula Ila or Formula IIb:

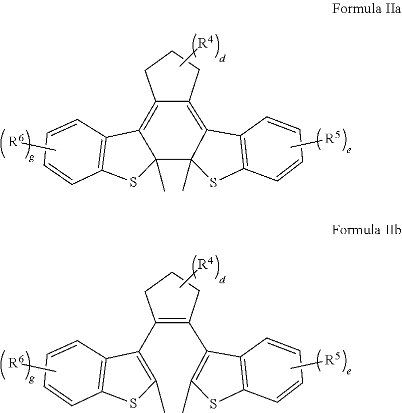

wherein
R⁴, R⁵, and R⁶ are each independently —C₁₋₆ alkyl, —NH₂, —NHCH₃, —N(CH₃)₂, —OH, —OCH₃, —OCH(CH₃)₂, —OCF₃, —CF₃, —Cl, —Br, or —F;
d is 0, 1, 2, 3, 4, 5, or 6; and
e and g are each independently 0, 1, 2, or 3.

In some embodiments, the compound of Formula Ila has a Amax of about 480 nm to about 680 nm. In some embodiments, the compound of Formula Ila has a λₘₐₓ of about 520 nm. In some embodiments, the compound of Formula IIb has a λₘₐₓ of about 225 nm to about 325 nm. In some embodiments, the compound of Formula IIb has a λₘₐₓ of about 265 nm.

In some embodiments, the diarylethene is

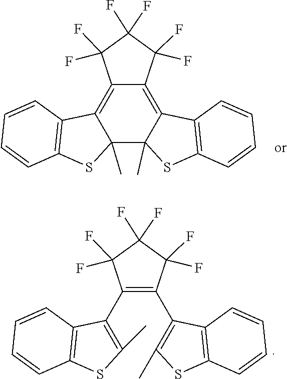

In some embodiments, the one or more photo-reactive components include a viologen. In some embodiments, the viologen a compound of Formula III, or a salt thereof:

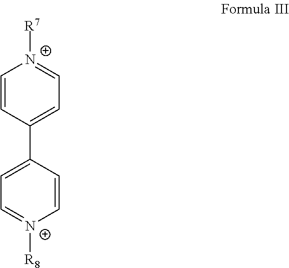

wherein
R⁷ and R⁸ are each independently —H or C₁₋₁₀ alkyl optionally substituted with —NH₂, —NHCH₃, —N(CH₃)₂, —OH, —OCH₃, —OCH(CH₃)₂, —OCF₃, —CF₃, —Cl, —Br, —F, or phenyl.

In some embodiments, the viologen, or a salt thereof, is

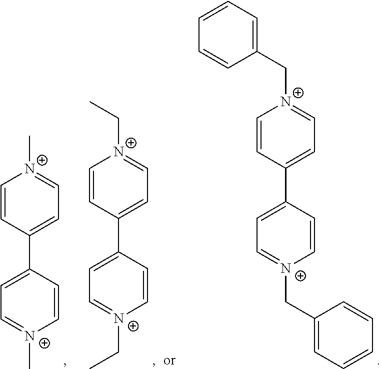 [SMILES: C[n+]1ccc(-c2cc[n+](C)cc2)cc1 ; CC[n+]1ccc(-c2cc[n+](CC)cc2)cc1 ; c1ccc(C[n+]2ccc(-c3cc[n+](Cc4ccccc4)cc3)cc2)cc1]

In some embodiments, the one or more photo-reactive components include a spiropyran. In some embodiments, the spiropyran is a compound of Formula IVa or Formula IVb:

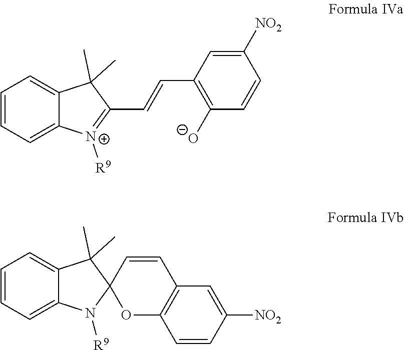

wherein R⁹ is independently —H or C₁₋₁₀ alkyl optionally substituted with —NH₂, —NHCH₃, —N(CH₃)₂, —OH, —OCH₃, —OCF₃, —CF₃, —OCH(CH₃)₂, —Cl, —Br, —F, or phenyl.

In some embodiments, the compound of Formula IVa has a λₘₐₓ of about 500 nm to about 600 nm. In some embodiments, the compound of Formula IVa has a λₘₐₓ of about 560 nm. In some embodiments, the compound of Formula IVb has a λₘₐₓ of about 250 nm to about 350 nm. In some embodiments, the compound of Formula IVb has a λₘₐₓ of about 300 nm.

In some embodiments, the spiropyran is

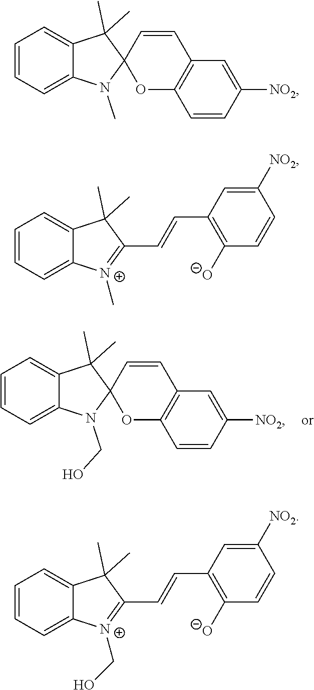

In some embodiments, the one or more photo-reactive components is an azobenzene. In some embodiments, the azobenzene is a compound of Formula Va or Formula Vb:

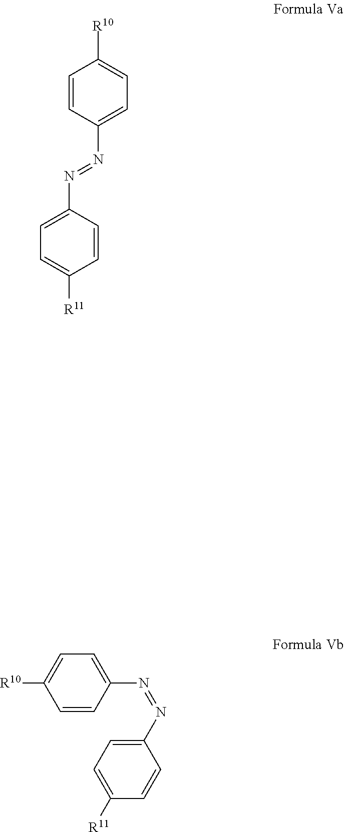

wherein
R¹⁰ and R¹¹ are each independently —H, —C₁₋₆ alkyl, —NH₂, —NHCH₃, —N(CH₃)₂, —OH, —OCH₃, —OCH(CH₃)₂, —OCF₃, —CF₃, —Cl, —Br, or —F.

In some embodiments, the compound of Formula Va has a λₘₐₓ of about 400 nm to about 500 nm. In some embodiments, the compound of Formula Va has a λₘₐₓ of about 420 nm to about 480 nm. In some embodiments, the compound of Formula Vb has a λₘₐₓ of about 300 nm to about 400 nm. In some embodiments, the compound of Formula Vb has a λₘₐₓ of about 350 nm to about 370 nm.

In some embodiments, the azobenzene, or configurational isomer thereof, is

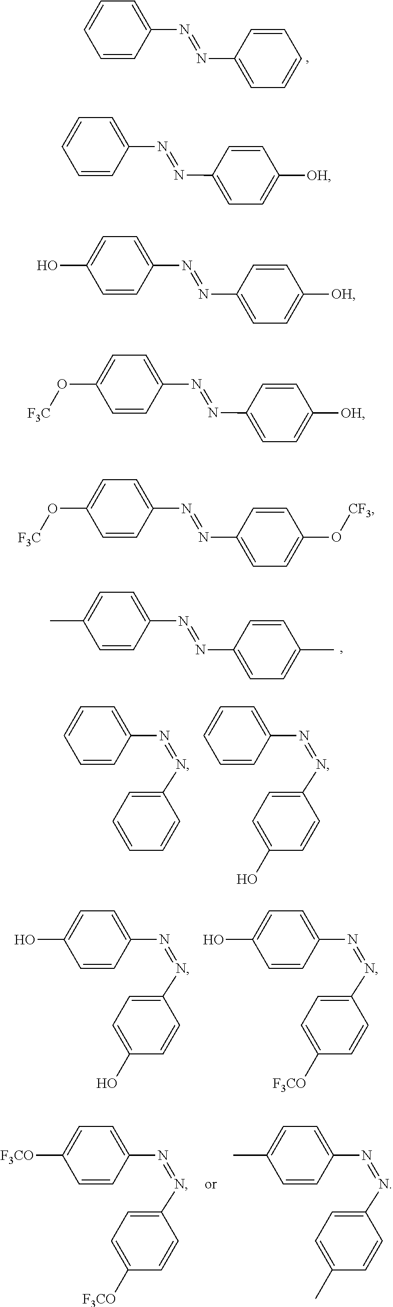

In some embodiments, the one or more photo-reactive components is a fulgide. In some embodiments, the fulgide is a compound of Formula VI, or a cyclized form thereof:

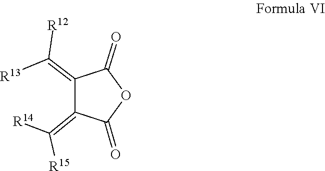

wherein
R¹², R¹³, R¹⁴, and R¹⁵ are each independently —H, —C₁₋₆ alkyl, phenyl, furanyl, or pyranyl;
   wherein phenyl, furanyl, or pyranyl of R¹², R¹³, R¹⁴, and R¹⁵ are each optionally substituted with one or two —H, —C₁₋₆ alkyl, —NH₂, —NHCH₃, —N(CH₃)₂, —OH, —OCH₃, —OCH(CH₃)₂, —OCF₃, —CF₃, —Cl, —Br, or —F;
wherein at least of R¹², R¹³, R¹⁴, and R¹⁵ is phenyl, furanyl, or pyranyl.

In some embodiments, the fulgide has a λₘₐₓ of about 325 nm to about 425 nm. In some embodiments, the fulgide has a λₘₐₓ of about 365 nm. In some embodiments, the cyclized fulgide has a λₘₐₓ of about 430 nm to about 530 nm. In some embodiments, the cyclized fulgide has a λₘₐₓ of about 490 nm.

In some embodiments, the fulgide is

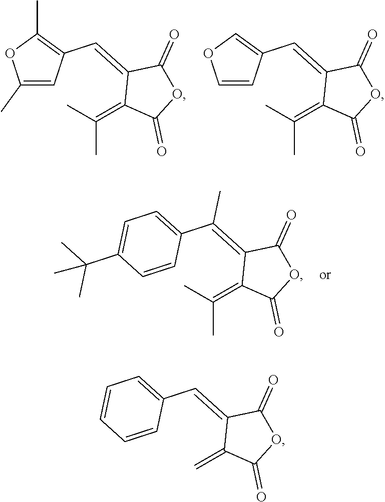

or a configurational isomer thereof.

In some embodiments, the cyclized fulgide is

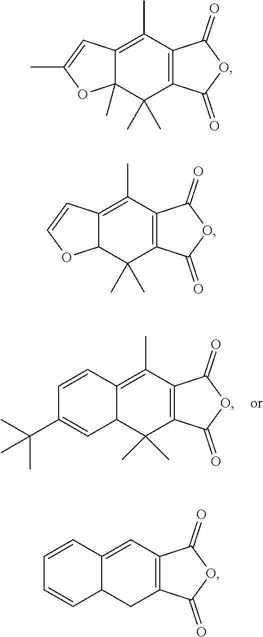

or a configurational isomer thereof.

In some embodiments, the one or more photo-reactive components include

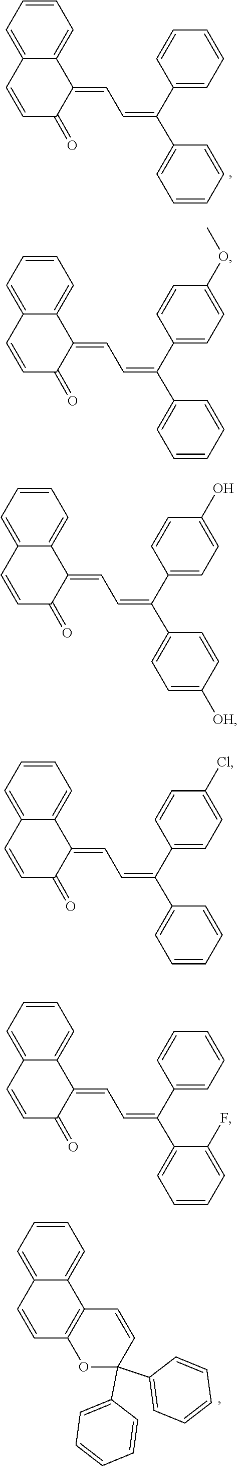

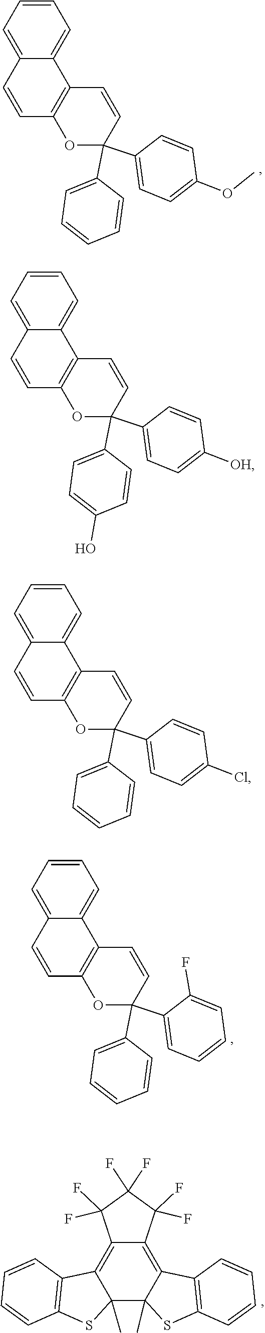

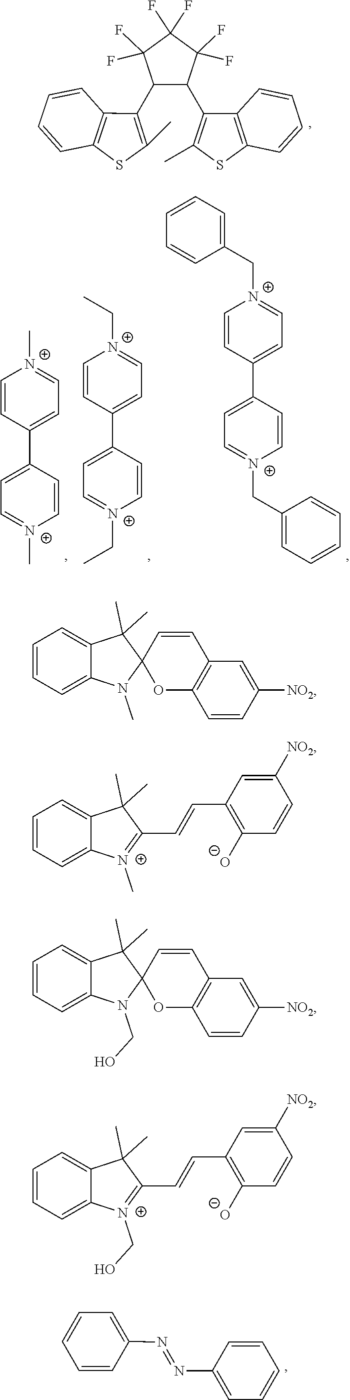

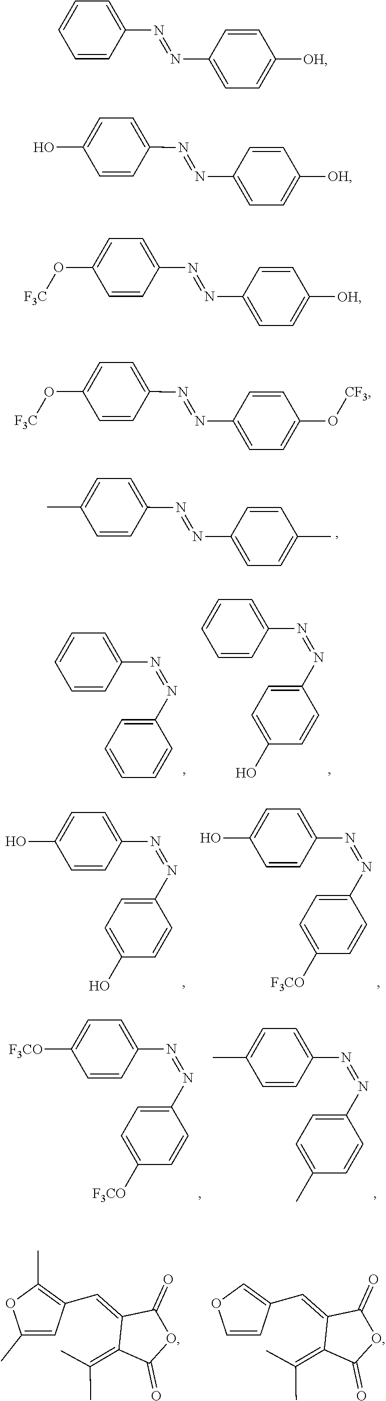

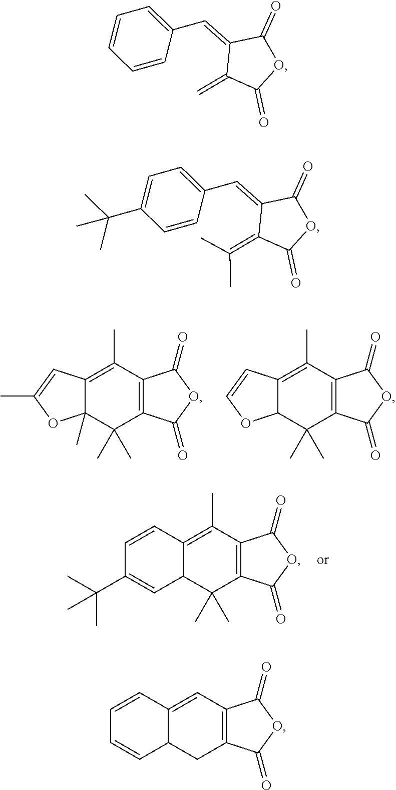

or a configurational isomer thereof or a salt thereof.

When exposed to an external stimulus (e.g., electromagnetic radiation) the photo-reactive component undergoes a conformational change. The change in conformational state can include a change from a cis to a trans configured double bond, rotating a molecular group about an axis, opening a hinged molecular group, bending a molecular chain, and unbending a molecular chain. The conformational change causes the coating to shift from a first conformational state to a second conformational state. When the coating is in the first state, the surface is characterized by a first property, and, when the coating is in the second state, the surface is characterized by a second property.

The surface of the photo-switchable polymer coatings disclosed herein change from a hydrophobic state to a hydrophilic state upon the conformational change. Since the reagents used in the sequencing process are mostly hydrophilic, the sequencing reactions take place only on the hydrophilic areas of the surface. As such, the hydrophobic surface is considered “locked” and the hydrophilic surface is considered “unlocked.”

The measure of the hydrophobicity or hydrophilicity of the surface can be determined by measuring the contact angle. A surface with a contact angle greater than or equal to 90° is considered hydrophobic, and a surface with a contact angle less than 90° is considered hydrophilic. The contact angle can be measured in various ways, including with an optical contact angle goniometer with this device, one uses an aqueous solution of citrate (0.1 M, pH 11.5) in air and then measures the contact angle using a goniometer (VCA-2500XE, AST) equipped with an electrometer (6517A, Keithley Inst.) and a carbon fiber microelectrode (Kation Scient). Contact angles can be averaged over at least 100 data points of multiple samples.

In some embodiments, the first state of the photo-switchable polymer coating is hydrophobic, and the second state of the photo-switchable polymer coating is hydrophilic. In some embodiments, the contact angle of the surface in the first conformation is greater than 90°, greater than 120°, or greater than 150°. In some embodiments, the contact angle of the surface in the first conformation is 91-100°, 100-110°, 110-120°, 120-130°, 130-140°, 140-150°, 150-160°, 160-170°, 170-180°. In some embodiments, the photo-switchable polymer coating includes poly(allylamine hydrochloride) modified with 3-(aminopropyl)triethoxysilane and 7-[(trifluoromethoxyphenylazo)phenoxy]pentanoic acid and the contact angle of the surface in the first conformation is 152°±3°.

In some embodiments, the contact angle of the surface in the second conformation is less than 90°, less than 30°, or less than 5°. In some embodiments, the contact angle of the surface in the second conformation is 1-10°, 10-20°, 20-30°, 30-40°, 40-50°, 50-60°, 70-80°, or 80-89°. In some embodiments, the photo-switchable polymer coating includes poly(allylamine hydrochloride) modified with 3-(aminopropyl)triethoxysilane and 7-[(trifluoromethoxyphenylazo)phenoxy]pentanoic acid and the contact angle of the surface in the second conformation is 5°±3°.

The surface can also include fiducial makers so that the locations that have undergone the conformation change can be tracked. Software can be used to store the spatial information.

In some embodiments, the coated substrate further includes a second coating wherein the second coating includes an additional component that shifts from a first conformation to a second conformation when exposed to an external stimulus. In some embodiments, the external stimulus is electromagnetic radiation, e.g., visible, ultraviolet, or infrared light. The external stimulus can include application of a voltage, a change in an applied voltage, a change in temperature or pH, exposure to a magnetic field, removal of a magnetic field, a change in capacitance, application or removal of an electrostatic charge, or any combination of the above. The operation of this mechanism is closely aligned with that of photo-stimulation. Initially, a layer of stimulus-responsive polymers is applied to the surface. Subsequently, a stimulus is directed to a specific locale; for example, an electromagnetic field is targeted at a minute region. Only the materials within this localized stimulated area will undergo a co-responsive bonding transformation, altering the surface properties from hydrophobic to hydrophilic. The newly hydrophilic regions are then suitable for undergoing SBS.

### Preparation Methods

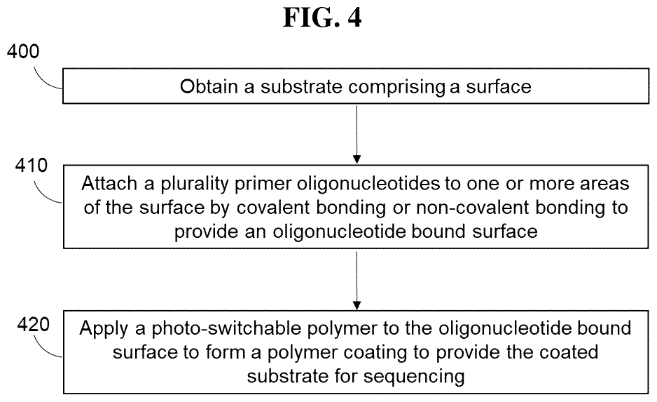

Also provided herein are methods of preparing a coated substrate. FIG. 4 is a flowchart illustrating an overview of the preparation methods consistent with some embodiments of the present disclosure. The methods can include obtaining a substrate comprising a surface (400), attaching primer oligonucleotides to the surface to form the oligonucleotide bound surface (410), and applying a photo-switchable polymer to the oligonucleotide bound surface to form the coated substrate for sequencing (420).

### Substrate

Obtaining the substrate can include exposing the surface to a plasma ashing in order to clean and activate the surface of the substrate. For example, the plasma ashing process can remove organic material and introduce surface hydroxyl groups. Other suitable cleaning processes can be used to clean the substrate, depending, in part, on the type of substrate. For example, chemical cleaning can be performed with oxidizing agents or caustic solutions.

The substrate can then be exposed to a process that will prepare the surface of the substrate for deposition of the functionalized polymer to form the functionalized polymer layer. In an example, the patterned substrate can be exposed to silanization, which attaches a silane or the silane derivative to the patterned wafer surface. Silanization introduces the silane or the silane derivative across the surface. In some embodiments, the silane or silane derivative is selectively introduced only to the depressions of a patterned substrate or to micro-locations (which are isolated from each other) of a non-patterned substrate. Silanization can be accomplished using any silane or silane derivative. The selection of the silane or silane derivative can depend, in part, upon the functionalized molecule that is to be used to form the functionalized polymer layer, as it can be desirable to form a covalent bond between the silane or silane derivative and the functionalized polymer layer. The method used to attach the silane or silane derivative to the substrate can vary depending upon the silane or silane derivative that is being used.

In some embodiments, the substrate surface is pre-treated with the (3-aminopropyl)triethoxysilane (APTES) or (3-aminopropyl)trimethoxysilane (APTMS) to covalently link silicon to one or more oxygen atoms on the surface (each silicon can bond to one, two or three oxygen atoms). This chemically treated surface is baked to form an amine group monolayer. The amine groups are then reacted with Sulfo-HSAB to form an azido derivative. ETV activation at 21 °C with 1 J/cm² to 30 J/cm² of energy generates an active nitrene species, which can readily undergo a variety of insertion reactions with PAZAM (e.g., the functionalized molecule).

Other silanization methods can be used. Examples of suitable silanization methods include vapor deposition, a YES® method, spin coating, or other deposition methods. Some examples of methods and materials that can be used to silanize the substrate are described herein, although it is to be understood that other methods and materials can be used.

In some embodiments, utilizing the YES® CVD oven, the patterned substrate is placed in the CVD oven. The chamber can be vented and then the silanization cycle started. During cycling, the silane or silane derivative vessel can be maintained at a suitable temperature (e.g., about 120 °C for norbornene silane), the silane or silane derivative vapor lines be maintained at a suitable temperature (e.g., about 125 °C for norborene silane), and the vacuum lines be maintained at a suitable temperature (e.g., about 145 °C).

In some embodiments, the silane or silane derivative (e.g., liquid norbornene silane) can be deposited inside a glass vial and placed inside a glass vacuum desiccator with a patterned substrate. The desiccator can then be evacuated to a pressure ranging from about 15 mTorr to about 30 mTorr and placed inside an oven at a temperature ranging from about 60 °C to about 125 °C. Silanization is allowed to proceed, and then the desiccator is removed from the oven, cooled, and vented in air.

Vapor deposition, the YES® method, and/or the vacuum desiccator can be used with a variety of silane or silane derivative, such as those silane or silane derivatives including examples of the unsaturated moieties disclosed herein. As examples, these methods can be used when the silane or silane derivative includes an alkene or cycloalkene unsaturated moiety, such as norborene, a norbornene derivative.

The silanized patterned wafer can then be exposed to a process that will form the functionalized polymer layer on the silanized depressions and silanized interstitial regions. The functionalized molecule can be present in a mixture. In some embodiments, the mixture includes PAZAM in water, or in an ethanol and water mixture. The functionalized polymer layer can be formed on the surface of the silanized patterned wafer (i.e., onto the silanized depressions and the silanized interstitial regions) using any suitable technique. The functionalized molecule can be deposited on the surface of the patterned substrate using spin coating, or dipping or dip coating, or flow of the functionalized molecule under positive or negative pressure, or other suitable techniques.

The attachment of the functionalized polymer layer to the silanized depressions and silanized interstitial regions can be through covalent bonding. The covalent linking of the functionalized polymer layer to the silanized depressions is helpful for maintaining the functionalized polymer layer in the depressions throughout the lifetime of the ultimately formed flow cell during a variety of uses. The following are some examples of reactions that can take place between the silane or silane derivative and the functionalized polymer layer.

When the silane or silane derivative includes norbornene or a norbornene derivative as the unsaturated moiety, the norbornene or a norbornene derivative can: i) undergo a 1,3-dipolar cycloaddition reaction with an azide/azido group of PAZAM; ii) undergo a coupling reaction with a tetrazine group attached to PAZAM; iii) undergo a cycloaddition reaction with a hydrazone group attached to PAZAM; iv) undergo a photo-click reaction with a tetrazole group attached to PAZAM; or v) undergo a cycloaddition with a nitrile oxide group attached to PAZAM.

When the silane or silane derivative includes cyclooctyne or a cyclooctyne derivative as the unsaturated moiety, the cyclooctyne or cyclooctyne derivative can: i) undergo a strain-promoted azide-alkyne 1,3-cycloaddition (SPAAC) reaction with an azide/azido of PAZAM, or ii) undergo a strain-promoted alkyne-nitrile oxide cycloaddition reaction with a nitrile oxide group attached to PAZAM.

When the silane or silane derivative includes a bicyclononyne as the unsaturated moiety, the bicyclononyne can undergo similar SPAAC alkyne cycloaddition with azides or nitrile oxides attached to PAZAM due to the strain in the bicyclic ring system.

In some embodiments, the patterned substrate is not exposed to silanization. Rather, the patterned substrate is exposed to plasma ashing, and then the functionalized polymer layer is directly spin coated (or otherwise deposited) on the plasma ashed patterned substrate. In this example, plasma ashing can generate surface-activating agent(s) (e.g., —OH groups) that can adhere the functionalized coating layer to the patterned substrate. In these examples, the functionalized polymer layer is selected so that it reacts with the surface groups generated by plasma ashing.

The process to form the functionalized polymer layer can include coating the patterned substrate or silanized substrate with a functionalized molecule and then exposing the functionalized molecule to a curing process to form the functionalized polymer layer across the entire patterned substrate (i.e., on depression(s) and interstitial region(s)). In an example, curing the functionalized molecule can take place at a temperature ranging from room temperature (e.g., about 25 °C) to about 95 °C for a time ranging from about 1 millisecond to about several days. In another example, the time can range from 10 seconds to at least 24 hours. In still another example, the time can range from about 5 minutes to about 2 hours.

The silanized and functionalized polymer coated patterned substrate can be exposed to a cleaning process. This process can utilize a water bath and sonication. The water bath can be maintained at a relatively low temperature ranging from about 22 °C to about 45 °C. In another example the water bath temperature ranges from about 25 °C to about 30 °C.

The silanized and coated patterned substrate is then exposed to polishing, if needed, to remove portion(s) of the functionalized polymer layer from the silanized interstitial regions. The portions of the silane or silane derivative that are adjacent to the interstitial regions can or cannot be removed as a result of polishing. When these silanized portions are completely removed, it is to be understood that the underlying substrate is exposed.

The polishing process can be performed with a gentle chemical slurry (including, e.g., an abrasive, a buffer, a chelating agent, a surfactant, and/or a dispersant) which can remove the thin functionalized polymer layer, and in some instances, at least part of the silane or silane derivative, from the interstitial regions without deleteriously affecting the underlying substrate at those regions. Alternatively, polishing can be performed with a solution that does not include the abrasive particles.

The chemical slurry can be used in a chemical mechanical polishing system to polish the surface of the silanized and functionalized polymer coated patterned substrate. The polishing head(s)/pad(s) or other polishing tool(s) is/are capable of polishing the functionalized polymer layer from the interstitial regions 6 while leaving the functionalized polymer layer in the depressions and leaving the underlying substrate at least substantially intact. In some embodiments, the polishing head is a Strasbaugh ViPRR II polishing head.

### Primer Oligonucleotides

The primer oligonucleotides can be attached to the surface of a substrate to form the oligonucleotide bound surface using any method known in the art, for example, such as those described in U.S. Pat. No. 8,895,249, WO 2008/093098, and U.S. Pat. Pub. No. 2011/0059865 A1, amongst others. In some embodiments, the primer oligonucleotides can include one or more unnatural or modified nucleic acids, unnatural backbone linkages, restriction enzyme sequences, or any combination thereof. The primer oligonucleotides are immobilized on the surface through hybridization of the adapter portion that is configured to bind to at least one primer oligonucleotide.

The primers can be directly attached to the surface. In some embodiments, the surface can have functional groups that can immobilize the terminal groups at the 5′ end of the primers. In some embodiments, immobilization can be by single point covalent attachment to the surface at the 5′ end of the primers. Any suitable covalent attachment means known in the art can be used. Examples of terminated primers that can be used include an alkyne terminated primer, a tetrazine terminated primer, an azido terminated primer, an amino terminated primer, an epoxy or glycidyl terminated primer, a thiophosphate terminated primer, a thiol terminated primer, an aldehyde terminated primer, a hydrazine terminated primer, a phosphoramidite terminated primer, and a triazolinedione terminated primer. In some embodiments, a succinimidyl (NHS) ester terminated primer is reacted with an amine on the surface, an aldehyde terminated primer is reacted with a hydrazine on the surface, an alkyne terminated primer is reacted with an azide on the surface, an azide terminated primer is reacted with an alkyne or DBCO (dibenzocyclooctyne) on the surface, an amino terminated primer is reacted with an activated carboxylate group or NHS ester on the surface, a thiol terminated primer is reacted with an alkylating reactant (e.g., iodoacetamine or maleimide) on the surface, a phosphoramidite terminated primer is reacted with a thioether on the surface, or a biotin-modified primer is reacted with streptavidin on the surface. The primers can also be attached to the surface by flow through deposition (e.g., using a temporarily bound lid), dunk coating, spray coating, or puddle dispensing.

The primers can also be grafted to the functionalized polymer layer in the depressions. In some embodiments, grafting is accomplished by dunk coating, spray coating, puddle dispensing, or by another suitable method that will attach the primers to the functionalized polymer layer in at least some of the depressions. Each of these example techniques can utilize the primer solution or mixture disclosed herein, which can include the primers, water, a buffer, and a catalyst.

### Photo-Switchable Polymer Coating

The photo-switchable polymer can be applied onto the oligonucleotide bound surface forming the polymer coating. The photo-switchable polymer application method can be performed entirely at the wafer level, entirely at the die level, in part at the wafer level, and/or in part at the die level. As an example of performing the method partially at the wafer and die levels, the method can be initiated using a wafer, which is then diced to form several dies, and the method can continue using each of the dies. The ability to perform open wafer processing (e.g., chemical or physical steps undertaken prior to lid bonding), at least in some embodiments, enables a variety of metrology/analytical techniques to be used for quality control and characterization. Prior to being bonded to form a flow cell, the patterned and surface modified wafer/substrate can be exposed to, for example, atomic force microscopy (AFM), scanning electron microscopy (SEM), ellipsometry, goniometry, scatterometry, and/or fluorescence techniques. Alternatively, the bonded flow cell can be exposed to these techniques. At the die level, the method(s) can be performed on an open-faced die, or on an assembled flow cell (with an enclosed flow channel).

After the photo-switchable polymer application, the photo-switchable polymer can be exposed to a curing process to cure the photo-switchable polymer across the entire substrate, e.g., patterned substrate (e.g., on depression(s) and interstitial region(s)). In some embodiments, curing takes place at a temperature ranging from room temperature (e.g., about 25 °C) to about 60 °C for a time ranging from about 5 minutes to about 2 hours.

To form the polymer coating in the depression(s) and not on the interstitial region(s) of a patterned substrate, the polymer coating can be polished off of the interstitial regions using i) a basic, aqueous slurry having a pH ranging from about 7.5 to about 11 and including abrasive particles or ii) a polishing pad and a solution free of abrasive particles.

The photo-switchable polymer can also be selectively deposited, or patterned, such that the surface chemistry is covered and such that a bonding region of the patterned substrate remains exposed. The bonding region of the patterned substrate is generally located on some of the interstitial region(s) and/or outer edges of the patterned substrate where a lid will be bonded to the patterned substrate. When the patterned substrate is a wafer, the bonding region can define the boundaries of several flow cells that are being formed from the wafer. When the patterned substrate is a die, the bonding region can define the outer boundaries of one flow cell that is being formed. It is to be understood that other portion(s) of the patterned substrate that are not part of the bonding region can be coated with the coating. Selectively depositing or patterning the coating can be accomplished via dip coating, spin coating, spray coating, ultrasonic spray coating, doctor blade coating, aerosol printing, or inkjet printing. A mask can be used to cover the bonding region of the patterned substrate so that the coating is not applied on the masked bonding region.

The photo-switchable polymer coating also includes the one or more photo-reactive components described above. The photo-switchable polymer coating can include the polymer material and one or more photo-reactive components described above. The coating can be deposited on the surface of a patterned substrate using spin coating or dip coating or other suitable techniques. The polymer material and one or more photo-reactive components can be present together in the solution used in the deposition method. In some embodiments, the polymer material is applied to the substrate prior to the completion of polymerization, and polymerization to form the coating is done on the substrate surface. In some embodiments, the polymer material is deposited onto the surface and then the one or more photo-reactive components are grafted to the polymer material deposited on the surface. In some embodiments, the one or more photo-reactive components are grafted to the polymer material prior to deposition on the surface.

Grafting can be accomplished by dunk coating, spray coating, puddle dispensing, or by another suitable method that will attach the one or more photo-reactive components to the polymer material in at least some, e.g., most of, a plurality of, or all, of the depressions.

Dunk coating can involve submerging the patterned substrate (having a coating layer in the depression(s) thereof) into a series of temperature-controlled baths. The baths can also be flow controlled and/or covered with a nitrogen blanket. The baths can include the one or more photo-reactive components. Throughout the various baths, the one or more photo-reactive components will attach to the polymer coating layer in at least some of the depression(s). In some embodiments, the coated and polished patterned substrate will be introduced into a first bath including the one or more photo-reactive components solution or mixture where a reaction takes place to attach the one or more photo-reactive components, and then the patterned substrate will be moved, e.g., dipped or dunked, into one or more additional baths for washing. The patterned substrate can be moved from bath to bath with a robotic arm or manually. A drying system can also be used in between baths and/or after dunk coating.

Spray coating can be accomplished by spraying the one or more photo-reactive component solutions or mixtures directly onto the coated and polished patterned substrate. The spray coated wafer can be incubated for a time ranging from about 4 minutes to about 60 minutes at a temperature ranging from about 0 °C to about 70 °C. After incubation, the primer solution or mixture can be diluted and removed using, for example, a spin coater.

Puddle dispensing can be performed according to a pool and spin-off method, and thus can be accomplished with a spin coater. The one or more photo-reactive component solutions or mixtures can be applied (manually or via an automated process) to the coated and polished patterned substrate. The applied one or more photo-reactive component solutions or mixtures can be applied to or spread across the entire surface of the coated and polished patterned substrate. The one or more photo-reactive component-coated, patterned substrates can be incubated for a time ranging from about 2 minutes to about 60 minutes at a temperature ranging from about 0 °C to about 80 °C. After incubation, the one or more photo-reactive component solutions or mixtures can be diluted and removed using, for example, the spin coater.

In some examples, after the one or more photo-reactive components are grafted to the polymer layer in the depression(s), this example of the method further includes applying the coating on the surface chemistry and on at least a portion of the substrate.

The application process of the photo-switchable polymer coating can also include drying. Drying can be accomplished via air exposure, nitrogen exposure, vacuum, heating (e.g., in an oven), or spin coating (i.e., spinning until dry).

### Sequencing Methods Using the Coated Substrates

This disclosure also provides methods of sequencing wherein the substrate used for sequencing is coated with a photo-switchable polymer coating in a locked state that is unlocked during use using a stimulus, such as electromagnetic radiation.

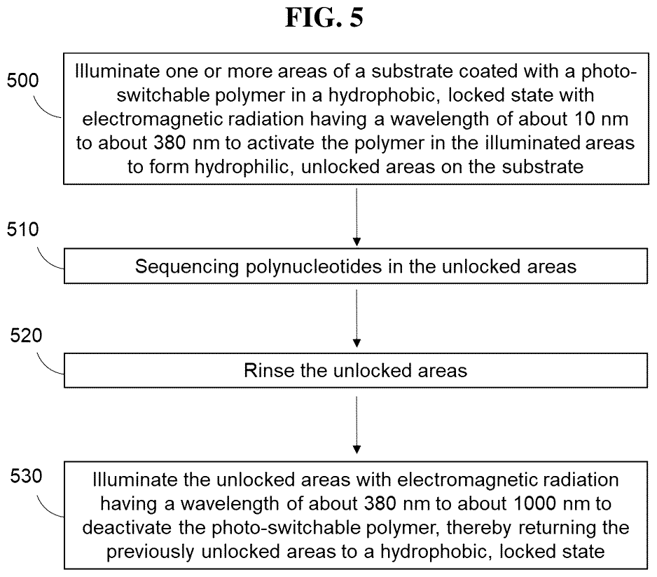

FIG. 5 is a flow chart illustrating an overview of sequencing methods consistent with some embodiments of the present disclosure. The methods can include illuminating one or more areas of a substrate coated with a photo-switchable polymer in a hydrophobic, locked state with electromagnetic radiation having a wavelength, for example, of about 260 nm to about 380 nm to activate the polymer in the illuminated areas to form hydrophilic, unlocked areas on the substrate (500); sequencing polynucleotides in the unlocked areas (510); rinsing the unlocked areas (520); and illuminating the unlocked areas with electromagnetic radiation having a wavelength, for example, of about 380 nm to about 1000 nm to deactivate the photo-switchable polymer, thereby returning the previously unlocked areas to a hydrophobic, locked state (530).

The disclosed method can include preparing DNA or RNA samples to be compatible with a sequencer. Sequencing libraries can be created by fragmenting DNA and adding specialized adapters to both ends. These adapters can contain complementary sequences that allow the DNA fragments to bind to the flow cell. Fragments can then be amplified and purified. Multiple libraries can be pooled together and sequenced in the same run. During adapter ligation, unique index sequences, or “barcodes,” are added to each library. These barcodes are used to distinguish between the libraries during data analysis.

After the libraries are prepared, the photo-switchable polymer can be switched from the locked state to the unlocked state using electromagnetic radiation, and the libraries can be loaded onto the unlocked areas of the flow cell and placed on the sequencer. The clusters of DNA fragments are amplified in a process called cluster generation, resulting in millions of copies of single-stranded DNA. Clustering can occur automatically. In some embodiments, DNA library prep is performed on the surface that has transposome. After DNA fragmentation and adaptor addition, the library prepared on the surface is ready for clustering.

The flow cells are then used to sequence the DNA. For example, a process called sequencing by synthesis (SBS) can be used. In SBS, chemically modified nucleotides can bind to the DNA template strand through natural complementarity. Each nucleotide includes a fluorescent tag and a reversible terminator that blocks incorporation of the next base. The fluorescent signal indicates which nucleotide has been added, and the terminator is cleaved so the next base can bind. After reading the forward DNA strand, the reads are washed away, and the process repeats for the reverse strand. Once completed, the phot-switchable polymer can be switched back into the locked state using electromagnetic radiation. After sequencing, software can be used to identify nucleotides.

### Illuminating Photo-Switchable Polymer

The external stimulus that causes the coating to shift from one conformational state to another can be a specific type and/or wavelength of electromagnetic radiation, such as light. For example, ultra-violet (UV) light can be used to shift the coating from a first conformational state to a second conformational state. Visible or infrared (IR) light can be used to shift the coating from the second conformational state back to the first conformational state. As discussed above, the photo-switchable polymer coating in the first conformational state can be is hydrophobic, and the photo-switchable polymer coating in the second conformational state can be hydrophilic. The wavelength of light used is generally dependent on the absorbance spectra of the photo-switchable polymer.

In some embodiments, the photo-switchable polymer coating includes poly(allylamine hydrochloride) modified with 3-(aminopropyl)triethoxysilane and 7-[(trifluoromethoxyphenylazo)phenoxy]pentanoic acid and is irradiated with light having a wavelength of about 365 nm to shift the coating from the first, hydrophobic conformational state to the second, hydrophilic conformational state. In some embodiments, the photo-switchable polymer coating includes poly(allylamine hydrochloride) modified with 3-(aminopropyl)triethoxysilane and 7-[(trifluoromethoxy-phenylazo)phenoxy]pentanoic acid and is irradiated with light having a wavelength of about 440 nm to shift the coating from the second, hydrophilic conformational state to the first, hydrophobic conformational state.

In some embodiments, the photo-switchable polymer coating includes

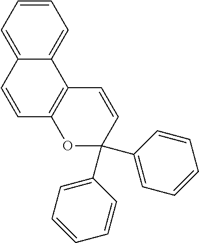 [SMILES: C1=CC(c2ccccc2)(c2ccccc2)Oc2ccc3ccccc3c21]

and is irradiated with light having a wavelength of about 320 nm to shift the coating from the first, hydrophobic conformational state to the second, hydrophilic conformational state. In some embodiments, the photo-switchable polymer coating includes

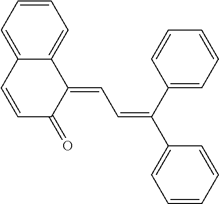 [SMILES: O=C1C=Cc2ccccc2/C1=C/C=C(c1ccccc1)c1ccccc1]

and is irradiated with light having a wavelength of about 430 nm to shift the coating from the second, hydrophilic conformational state to the first, hydrophobic conformational state.

In some embodiments, the photo-switchable polymer coating includes

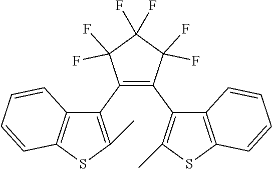 [SMILES: Cc1sc2ccccc2c1C1=C(c2c(C)sc3ccccc23)C(F)(F)C(F)(F)C1(F)F]

and is irradiated with light having a wavelength of about 265 nm to shift the coating from the first, hydrophobic conformational state to the second, hydrophilic conformational state. In some embodiments, the photo-switchable polymer coating includes

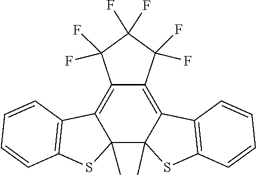 [SMILES: CC12Sc3ccccc3C1=C1C(=C3c4ccccc4SC32C)C(F)(F)C(F)(F)C1(F)F]

and is irradiated with light having a wavelength of about 520 nm to shift the coating from the second, hydrophilic conformational state to the first, hydrophobic conformational state.

In some embodiments, the photo-switchable polymer coating includes

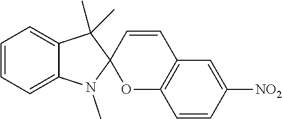 [SMILES: CN1c2ccccc2C(C)(C)C12C=Cc1cc([N+](=O)[O-])ccc1O2]

and is irradiated with light having a wavelength of about 300 nm to shift the coating from the first, hydrophobic conformational state to the second, hydrophilic conformational state. In some embodiments, the photo-switchable polymer coating includes

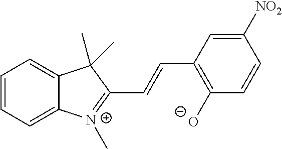 [SMILES: C[N+]1=C(/C=C/c2cc([N+](=O)[O-])ccc2[O-])C(C)(C)c2ccccc21]

and is irradiated with light having a wavelength of about 560 nm to shift the coating from the second, hydrophilic conformational state to the first, hydrophobic conformational state.

In some embodiments, the photo-switchable polymer coating includes

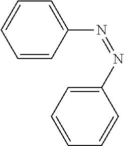 [SMILES: c1ccc(/N=N\c2ccccc2)cc1]

and is irradiated with light having a wavelength of about 360 nm to shift the coating from the first, hydrophobic conformational state to the second, hydrophilic conformational state. In some embodiments, the photo-switchable polymer coating includes

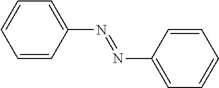 [SMILES: c1ccc(/N=N/c2ccccc2)cc1]

and is irradiated with light having a wavelength of about 450 nm to shift the coating from the second, hydrophilic conformational state to the first, hydrophobic conformational state.

In some embodiments, the photo-switchable polymer coating includes

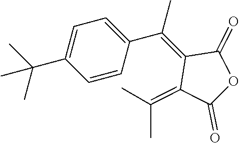 [SMILES: CC(C)=C1C(=O)OC(=O)/C1=C(\C)c1ccc(C(C)(C)C)cc1]

and is irradiated with light having a wavelength of about 365 nm to shift the coating from the first, hydrophobic conformational state to the second, hydrophilic conformational state. In some embodiments, the photo-switchable polymer coating includes

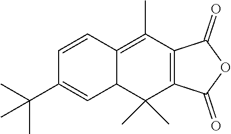 [SMILES: CC1=C2C=CC(C(C)(C)C)=CC2C(C)(C)C2=C1C(=O)OC2=O]

and is irradiated with light having a wavelength of about 490 nm to shift the coating from the second, hydrophilic conformational state to the first, hydrophobic conformational state.

The coating can be irradiated with light for at least about 1, 2, 3, 4, 5, 6, 7, 8, 9, or 10 ten minutes, or more. The entire surface area of the coating can be irradiated with light. In some embodiments, a portion, e.g., a specific portion, of the surface area of the coating is irradiated with light. In some embodiments, the conformational state of the photo-switchable polymer coating is changed from a first to a second state in the area that is irradiated with light. In some embodiments, the conformational state of the photo-switchable polymer coating is changed from a first to a second state beyond the areas that is irradiated with light.

The light source used for the irradiation can be, for example, a laser or an LED. Suitable lasers include the lasers listed in the table below.

| Laser | Type | Wavelength | |
|---|---|---|---|
| ArF, Krf, | Gas (excimer) | 193 nm, 248 nm, | |
| XeCl, Xef | | 308 nm, 353 nm | |
| Nitrogen | Gas | 337 | nm |
| Dye | Liquid | 400-1000 | nm |
| GaN | Semiconductor | 410 | nm |
| Argon-ion | Gas | 488 | nm |
| HeNe | Gas | 632.8 | nm |
| AlGalnP, | Semiconductor | 630-900 | nm |
| AlGaAs | | | |
| Ti:Saph | Solid-state | 650-1100 | nm |
| Yb:YAG | Solid-state | 1030 | nm |
| Yb-glass | Fiber | 1030 | nm |
| Nd:YAG | Solid-state | 1060 | nm |
| ND:glass | Fiber | 1060 | nm |
| InGaAs, | Semiconductor | 1100-2000 | nm |
| InGaAsP | | | |

When the external stimulus is applied to the surface and the conformational state of the photo-switchable polymer coating is changed from a first to a second state, a sufficient number of the photo-reactive components should change their conformation to change the surface properties of the coating. At least 60%, preferably 75%, and more preferably 90%, of the photo-reactive components should change their conformation in response to an external stimulus. The change in conformational state should be reversible. After reversing the conformational state of the coating at least 60%, preferably 75%, and more preferably 90% of the photo-reactive components should have the original conformation.

### Sequencing

Once a surface is “unlocked,” sequencing reactions can be performed. The process can also include amplification of template polynucleotide, which includes the process of amplifying or increasing the numbers of a template polynucleotide and/or of a complement thereof, by producing one or more copies of the polynucleotide and/or or its complement on the surface. Amplification can be carried out by a variety of known methods under conditions including, but not limited to, thermocycling amplification or isothermal amplification. For example, methods for carrying out amplification are described in U.S. Pat. Pub. No. 2009/0226975; WO 98/44151; WO 00/18957; WO 02/46456; WO 06/064199; and WO 07/010251.

Briefly, amplification can occur on the surface to which the template polynucleotide are immobilized. This type of amplification can be referred to as solid phase amplification, which when used in reference to template polynucleotide, refers to any template polynucleotide amplification reaction carried out on or in association with a surface. Typically, all or a portion of the amplified products are synthesized by extension of a primer that is immobilized on the surface.

Solid-phase amplification can include a template polynucleotide amplification reaction including only one species of primer oligonucleotide immobilized to a surface. Alternatively, the surface can include a plurality of first and second different immobilized primer oligonucleotide species. Solid phase template polynucleotide amplification reactions generally include at least one of two different types of nucleic acid amplification, interfacial or surface (or bridge) amplification. For instance, in interfacial amplification, the surface includes a template polynucleotide that is indirectly immobilized to the solid support by hybridization to an immobilized primer oligonucleotide, wherein the immobilized primer oligonucleotide is extended in the course of a polymerase-catalyzed, template-directed elongation reaction (e.g., primer extension), to generate an immobilized polynucleotide that remains attached to the solid support. After the extension phase, the polynucleotides (e.g., template polynucleotide and its complementary product) can be denatured such that the template polynucleotide is released into solution and made available for hybridization to another immobilized primer. The template polynucleotide can be made available in 1, 2, 3, 4, 5 or more rounds of primer extension or can be washed out of the reaction after 1, 2, 3, 4, 5 or more rounds of primer extension.

In surface (or bridge) amplification, an immobilized template polynucleotide hybridizes to a primer oligonucleotide. The 3′ end of the immobilized template polynucleotide provides the template for a polymerase-catalyzed, template-directed elongation reaction (e.g., primer extension) extending from the immobilized primer oligonucleotide. The resulting double-stranded product “bridges” the two primer oligonucleotides and both strands are covalently attached to the support. In the next cycle, following denaturation that yields a pair of single strands (the immobilized template polynucleotide and the extended-primer product) immobilized to the surface, both immobilized strands can serve as templates for new primer extension. Examples of bridge amplification can be found in U.S. Pat. Nos. 7,790,418; 7,972,820; WO 2000/018957; U.S. Pat. No. 7,790,418; and Adessi et al., Nucleic Acids Research (2000): 28(20): E87).

In some embodiments, after bridge amplification and while the double-stranded bridge complex exists, the surface can be treated with an exonuclease. The exonuclease will remove at least a portion of primer oligonucleotides that are not participating in a double-stranded bridge structure. The exonuclease can completely remove individual primer oligonucleotides or remove portions of individual primer oligonucleotides. Treating the surface with an exonuclease prior to applying the sequencing methods of the present disclosure can result in a lower background signal during sequencing.

Any suitable exonuclease can be used. Examples of suitable exonucleases include Exonuclease I, Exonuclease T, and Exonuclease VII (all are available from New England Biolabs, MA). Preferably, the exonuclease has a high specificity for single stranded DNA over double stranded DNA.

Amplification is used to produce colonies of immobilized polynucleotide templates. For example, the methods can produce clustered arrays of template polynucleotide colonies, analogous to those described in U.S. Pat. Nos. 7,115,400; 7,985,565; WO 00/18957; and WO 98/44151. “Clusters” and “colonies” are used interchangeably and refer to a plurality of copies of a polynucleotide template having the same sequence and/or complements thereof attached to a surface. Typically, the cluster includes a plurality of copies of a polynucleotide template having the same sequence and/or complements thereof, attached via their 5′ end to the surface. The copies of polynucleotide templates making up the clusters can be in a single or double stranded form.

The plurality of template polynucleotides can be in a cluster, each cluster containing template polynucleotides of the same sequence. A plurality of clusters can be sequenced, each cluster comprising template polynucleotides of the same sequence. Optionally, the sequence of the template polynucleotides in a first cluster is different from the sequence of the template polynucleotides of a second cluster. Optionally, the cluster is formed by annealing a template polynucleotide to a primer on a surface and amplifying the template polynucleotide under conditions to form the cluster that includes the plurality of template polynucleotides of the same sequence. Amplification can be thermal or isothermal.

Each colony can include a plurality of template polynucleotides of the same sequences. In some embodiments, the sequence of the template polynucleotides of one colony is different from the sequence of the template polynucleotides of another colony. Thus, each colony includes template polynucleotides having different target nucleic acid sequences. All the immobilized template polynucleotides in a colony are typically produced by amplification of the same template polynucleotide. In some embodiments, it is possible that a colony of immobilized template polynucleotides includes one or more primers without an immobilized template polynucleotide to which another polynucleotide having a different sequence can bind upon additional application of solutions containing free or unbound template polynucleotides.

The strand of the primer oligonucleotides can be cleaved in an adapter region of a template polynucleotide or can be cleaved in a region of the primer oligonucleotide (amplification primer) to which the template polynucleotide is bound.

The sequencing methods can be carried out on template polynucleotides that have been immobilized to a surface and amplified as described above. In some embodiments, the sequencing includes sequencing a single-stranded polynucleotide. In some embodiments, the double-stranded, now primer, polynucleotide can be denatured, and the cleaved strand can be washed away, leaving a single strand hybridized to the surface primer. Sequencing can occur using the surface primer and the remaining hybridized single strand. In some embodiments, the sequencing includes sequencing a strand of a double-stranded polynucleotide. For example, following cleavage and generation of the surface primer, sequencing can occur without removal of the cleaved strand. Sequencing of the strand of the double-stranded polynucleotide can proceed via strand displacement, nick translation, or any other suitable mechanism.

The sequencing methods of the present disclosure preferably use sequencing by synthesis (SBS) to elucidate the nucleotide sequence of regions of interest on the polynucleotide templates. SBS techniques include, but are not limited to, the Genome Analyzer systems (Illumina Inc., San Diego, CA) and the True Single Molecule Sequencing (tSMS)™ systems (Helicos BioSciences Corporation, Cambridge, MA). In the SBS technique, a number of sequencing-by-synthesis reactions are used to elucidate the identity of a plurality of bases at target positions within a target sequence.

In conventional SBS, these reactions rely on the use of a target nucleic acid sequence having at least two domains; a first domain to which a sequencing primer will hybridize; and an adjacent second domain, for which sequence information is desired. When SBS is used in conjunction with the sequencing methods of the current disclosure, a primer attached to the surface derived from the primer oligonucleotides (e.g., surface primer) is the sequencing primer and the second domain is the target nucleic acid sequence and/or other sequences of the template polynucleotide such as indexes. As will be described in detail below, at least a portion of the template polynucleotide (e.g., at least a portion of the adapter) can be already hybridized to the surface primer. Because the surface primer is serving as the sequencing primer, no additional sequencing primer is needed. This can allow for a reduction in the number of sequencing reagents. With the reduction in the number of sequencing reagents, the methods of the present disclosure can be more economically and environmentally friendly.

After formation of an initial sequencing complex (a template polynucleotide strand hybridized to a surface primer) as described above, a chain extension enzyme can be used to add deoxynucleotide triphosphates (dNTPs) to the surface sequencing primer, and each addition of dNTPs can be read to determine the identity of the added dNTP. This can proceed for many cycles. The sequence for which the nucleotide identity is determined is generally termed a “read.” Read lengths can be greater than 5, greater than 10, greater than 20, greater than 50, greater than 100, greater than 200, greater than 300, or greater than 400 nucleotides in length.

In some SBS embodiments, the template polynucleotide is hybridized with a surface primer and incubated in the presence of a polymerase and one or more labeled nucleotides that include a 3′ blocking group. Examples of labeled nucleotides that include a blocking group can be found in WO 2004/018497. The surface primer is extended such that the labeled nucleotide is incorporated. The presence of the blocking group permits only one round of incorporation, that is, the incorporation of a single nucleotide. The presence of the label permits identification of the incorporated nucleotide. In some embodiments, the label is a fluorescent label. A plurality of homogenous single nucleotide bases can be added during each cycle, such as used in the True Single Molecule Sequencing (tSMS)™ systems (Helicos BioSciences Corporation, Cambridge, MA).

Alternatively, all four nucleotide bases can be added during each cycle simultaneously, such as used in Genome Analyzer systems (Illumina Inc., San Diego, CA), particularly when each base is associated with a distinguishable label. After identifying the incorporated nucleotide by its corresponding label, both the label and the blocking group can be removed, thereby allowing a subsequent round of labeled nucleotide incorporation and identification. Determining the identity of the added nucleotide base includes, in some embodiments, repeated exposure of the newly added labeled bases to a light source that can induce a detectable emission due the addition of a specific nucleotide. In some embodiments, the label is a fluorescent label.

In some embodiments, the nucleotides used in SBS do not include a label, for example when pyrosequencing is used. Pyrosequencing detects the release of inorganic pyrophosphate (PPi) as particular nucleotides are incorporated into a nascent nucleic acid strand (Ronaghi, et al., Analytical Biochemistry 242 (1), 84-9 (1996); Ronaghi, Genome Res. 11 (1), 3-11 (2001); Ronaghi et al. Science 281 (5375), 363 (1998); U.S. Pat. Nos. 6,210,891; 6,258,568 and 6,274,320). In pyrosequencing, released PPi can be detected by being immediately converted to adenosine triphosphate (ATP) by ATP sulfurylase, and the level of ATP generated can be detected via luciferase-produced photons. Thus, the sequencing reaction can be monitored via a luminescence detection system. Excitation radiation sources used for fluorescence-based detection systems are not necessary for pyrosequencing procedures. Because the incorporation of any dNTP into a growing chain releases pyrophosphate, the four dNTP bases must be added to the system in separate steps.

Useful fluidic systems, detectors, and procedures that can be used for application of pyrosequencing to arrays of the present disclosure are described, for example, in WO2012058096A1; U.S. Pat. Pub. No. 2005/0191698 A1; U.S. Pat. Nos. 7,595,883; and 7,244,559.

Sequencing-by-ligation methods, such as those described in Shendure et al., Science, 309:1728-1732 (2005); U.S. Pat. Nos. 5,599,675; and 5,750,341, can also be used. Some embodiments can include sequencing-by-hybridization procedures as described, for example, in Bains et al., Journal of Theoretical Biology, 135 (3), 303-7 (1988); Drmanac et al., Nature Biotechnology 16, 54-58 (1998); Fodor et al., Science 251 (4995), 767-773 (1995); and WO 1989/10977. In both sequencing-by-ligation and sequencing-by-hybridization procedures, template nucleic acids (e.g., a target nucleic acid or amplicons thereof) that are present at sites of an array are subjected to repeated cycles of oligonucleotide delivery and detection. Fluidic systems for SBS methods can be readily adapted for delivery of reagents for sequencing-by-ligation or sequencing-by-hybridization procedures. Typically, the oligonucleotides are fluorescently labeled and can be detected using fluorescence detectors similar to those described with regard to SBS procedures herein or in references cited herein.

Some embodiments can use methods involving the real-time monitoring of DNA polymerase activity. For example, nucleotide incorporations can be detected through fluorescence resonance energy transfer (FRET) interactions between a fluorophore-bearing polymerase and γ-phosphate-labeled nucleotides, or with zeromode waveguides (ZMWs). Techniques and reagents for FRET-based sequencing are described, for example, in Levene et al. Science 299, 682-686 (2003); Lundquist et al. Opt. Lett. 33, 1026-1028 (2008); Korlach et al. Proc. Natl. Acad. Sci. USA 105, 1176-1181 (2008).

Some SBS embodiments include detection of a proton released upon incorporation of a nucleotide into an extension product. For example, sequencing based on detection of released protons can use an electrical detector and associated techniques that are commercially available from Ion Torrent (Guilford, Conn., a Life Technologies subsidiary) or sequencing methods and systems described in U.S. Pat. Nos. 8,262,900; 7,948,015; U.S. Pat. Pub. 2010/0137143 A1; or U.S. Pat. No. 8,349,167.

In addition, the sequencing methods described herein can be particularly useful for sequencing from an array clusters of template polynucleotides, where multiple sequences can be read simultaneously from multiple clusters on the array since each nucleotide at each position can be identified based on its identifiable label. Examples of such methods are described in U.S. Pat. Nos. 7,754,429; 7,785,796; and 7,771,973.

In some embodiments, where the template polynucleotides include one or more index sequences, the index sequences can be sequenced using SBS.

In some embodiments, SBS involves several rounds of incorporation of nucleotides for which the identities of the incorporated nucleotides are not determined. Such rounds of incorporation can be referred to as “dark cycles.” Dark cycling involves the sequential incorporation of nucleotides containing a 5′ blocking group and subsequent blocking group removal. Dark cycles can be used to skip the reading of index sequences, universal sequences, and/or any other sequence where the identity is not desired to be determined. Each dark cycle includes the incorporation of a nucleotide. Any suitable number of dark cycles of incorporation can be performed to effectively reach the portion of the template polynucleotide where determining the nucleotide sequence is desired. For example, 2 to 150 dark cycles can be performed, such as 3 to 100, 5 to 50, or 6 to 25 dark cycles. The sequence of the template polynucleotide strand to which the extended surface primer is complementary during the dark cycles is preferably known. Once the appropriate number of dark cycles of incorporation are performed, SBS (determining the identity of the nucleotides incorporated in subsequent cycles) can be performed.

After completion of the sequencing, the unlocked areas can be rinsed. The rinsing can include water.

Throughout this application, various publications are referenced. The disclosures of these publications in their entireties are hereby incorporated by reference into this application.

### EXAMPLES

### Example 1

In a first step, we obtain a Hiseq X flow cell, and added a lawn of primer oligonucleotides DNA P5/P7. The flow cells include several flow channels/lanes defined on a patterned silicon and/or a tantalum oxide substrate, where each lane is in fluid communication with a plurality of wells.

Next, we coat a protective, photo-switchable polymer azo-benene onto a surface of the flow cell. This provides a “locked” flow cell. In particular, we prepare solutions of poly(allylamine hydrochloride) modified with 3-(aminopropyl)triethoxysilane and 7-[(trifluoromethoxy-phenylazo)phenoxy]pentanoic acid with different concentrations. A flow through process is used to introduce one of the solutions into one of the lanes of each of the flow cells. The solution is dried to form a protective coating. These flow cells are dried using nitrogen gas. The dried test flow cells are then stored for 3 days at 60 °C. (equivalent to dry storage for 1 month at 25 °C or ambient conditions) with the coating in place.

To unlock the polymer coating of the flow cell after storage, we irradiate the back of the flow cell to ultra-violet light at a wavelength of about 365 nm for 10 minutes in the desired areas of the test flow cells. Then another HPTET quality control assay is performed in each of the lanes of each of the test and comparative flow cells. The HP-TET retention rate results are calculated using the before coating and after storage HP-TET results.

Thereafter, we run the flow cells through an Illumina Hiseq sequencer, using the following standard 300 cycles run for sequencing by synthesis.

Next, the surface of the flow cell is rinsed. Thereafter, we expose the backside of the flow cell to visible light to lock the flow cell again.

Then, another cycle of irradiation with UV light activates or unlocks additional regions on the flow cell, for additional SBS analysis.

A first HP-TET quality control assay is performed in each of the lanes of each of the example flow cell before the coatings were added to the example flow cells. HP or hairpin defines the secondary structure part of the DNA molecule used to probe the primers on the grafted flow cell surface, and TET (or TET+DNA) is a dye labeled oligonucleotide having complementary sequence to the primers used. TET is hybridized to the primers, the excess TET was washed away, and the fluorescence of the attached dye is measured by fluorescence detection.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A coated substrate for sequencing polynucleotide molecules, comprising:
primer oligonucleotides attached to a surface of a substrate; and
a coating on the surface, wherein the coating comprises one or more components that shift from a first conformation to a second conformation when irradiated with electromagnetic radiation in the wavelength range of 280 nm to 700 nm and wherein the first conformation causes the coating to be hydrophobic and the second conformation causes the coating to be hydrophilic.

2. The coated substrate of claim 1, wherein the electromagnetic radiation comprises ultra-violet (UV), visible, or infrared (IR) light.

3. The coated substrate of claim 1, wherein the coating comprises a polymer.

4. The coated substrate of claim 2, wherein the polymer comprises a polysaccharide, a poly(allylamine hydrochloride), a polyethylene glycol, or a polystyrene, or a combination of any two or more thereof.

5. The coated substrate of any one of claims 1-4, wherein the one or more components comprise a naphthopyran, a diarylethene, a viologen, a spiropyran, an azobenzene, or a fulgide, or a combination of any two or more thereof.

6. The coated substrate of any one of claims 1-4, wherein the one or more components comprise: 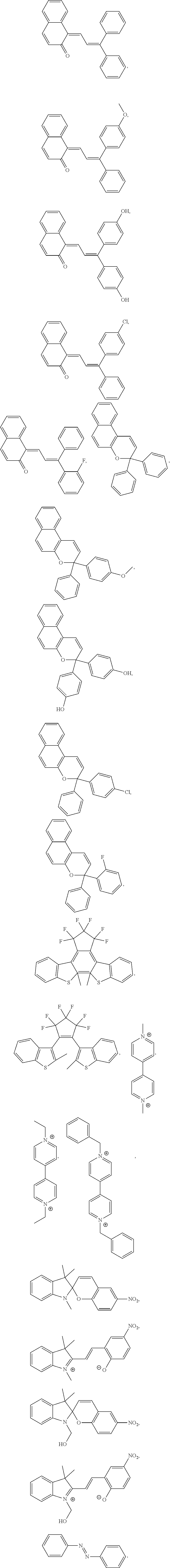 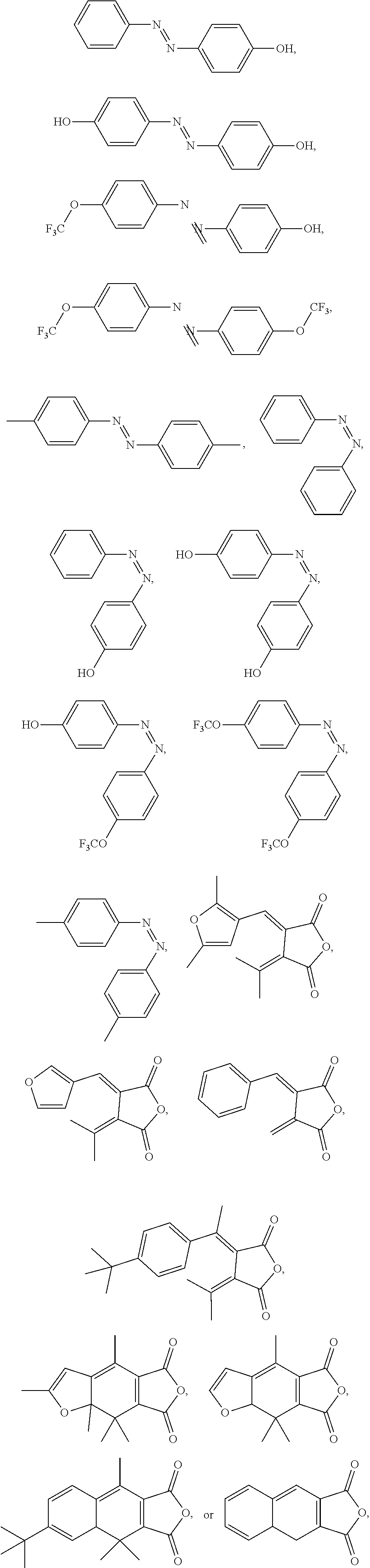
or a configurational isomer thereof, or a salt thereof.

7. The coated substrate of any one of claims 1-6, wherein the contact angle of the coating with the one or more components in the first conformation is great than 90°, great than 120°, or great than 150°.

8. The coated substrate of any one of claims 1-6, wherein the contact angle of the coating with the one or more components in the second conformation is less than 90°, less than 30°, or less than 5°.

9. The coated substrate of any one of claims 1-8, wherein the one or more components shifts from a first conformation to a second conformation when illuminated with UV light.

10. The coated substrate of any one of claims 1-9, wherein the thickness of the coating ranges from about 1 μm to about 15 μm.

11. The coated substrate of any one of claims 1-10, wherein the coated substrate for sequencing polynucleotide molecules is part of a segmented random access flow cell.

12. The coated substrate of any one of claims 1-11, wherein the coating is present on a portion of the surface.

13. The coated substrate of any one of claims 1-11, wherein the coating is present on the entirety of the surface.

14. The coated substrate of anyone of claims 1-13, wherein the coated substrate further includes a second coating wherein the second coating includes an additional component that shifts from a first conformation to a second conformation when exposed to an external stimulus.

15. The coated substrate of any one of claims 1-14, wherein the primer oligonucleotides comprise TTTTTTTTTTAATGATACGGCGACCACCGAGAUCTACAC (SEQ ID NO: 1) or TTTTTTTTTTCAAGCAGAAGACGGCATACGA (8-oxo G) AT (SEQ ID NO: 2).

16. A method of preparing a coated substrate for sequencing polynucleotide molecules, the method comprising:
obtaining a substrate comprising a surface;
attaching a plurality of primer oligonucleotides to one or more areas of the surface by covalent bonding or non-covalent bonding to provide an oligonucleotide bound surface; and
applying a photo-switchable polymer to the oligonucleotide bound surface forming a polymer coating to provide the coated substrate for sequencing.

17. The method of claim 16, wherein the primer oligonucleotides are immobilized on the surface through hybridization.

18. The method of claim 16 or 17, wherein applying the photo-switchable polymer to the templated substrate comprises spin coating, precision nozzle dispersion, or drop coating.

19. The method of any one of claims 16-18, further comprising curing the photo-switchable polymer.

20. The method of any one of claims 16-19, further comprising polishing or planarizing the coated substrate.

21. A method of sequencing a polynucleotide, the method comprising:
illuminating one or more areas of a substrate coated with a photo-switchable polymer in a hydrophobic, locked state with electromagnetic radiation having a wavelength of about 260 nm to about 380 nm to activate the polymer in the illuminated areas to form hydrophilic, unlocked areas on the substrate;
sequencing polynucleotides in the unlocked areas;
rinsing the unlocked areas; and
illuminating the unlocked areas with electromagnetic radiation having a wavelength of about 380 nm to about 1000 nm to deactivate the photo-switchable polymer, thereby returning the previously unlocked areas to a hydrophobic, locked state.

22. The method of claim 21, wherein the method comprises illuminating the entire area of the substrate coated with the photo-switchable polymer.

23. The coated substrate of claim 21, wherein the contact angle of the coating with the photo-switchable polymer in a hydrophobic, locked state is great than 90°, great than 120°, or great than 150°.

24. The coated substrate of any one of claims 21-23, wherein the contact angle of the coating with the photo-switchable polymer in a hydrophilic, unlocked state is less than 90°, less than 30°, or less than 5°.

25. The method of any one of claims 21-24, wherein illuminating one or more areas of a substrate coated with a photo-switchable polymer in a hydrophobic, locked state is done with electromagnetic radiation having a wavelength of about 300 nm to about 380 nm.

26. The method of any one of claims 21-25, wherein illuminating the unlocked areas are done with electromagnetic radiation having a wavelength of about 380 nm to about 780 nm.

27. The method of any one of claims 21-26, wherein the photo-switchable polymer coating comprises poly(allylamine hydrochloride) modified with 3-(aminopropyl)triethoxysilane and 7-[(trifluoromethoxy-phenylazo)phenoxy]pentanoic acid.

28. The method of claim 27, wherein the unlocked areas are illuminate with electromagnetic radiation having a wavelength of about 440 nm.

29. The method of any one of claims 21-28, wherein the sequencing of the polynucleotides comprises sequencing by synthesis.

30. The method of any one of claims 21-29, wherein rinsing the unlocked areas comprises rinsing with water.

31. A method of sequencing a polynucleotide, the method comprising:
illuminating one or more areas of a coated substrate of any one of claims 1-15 with electromagnetic radiation having a wavelength of about 260 nm to about 380 nm to activate the coated substrate in the illuminated areas to form hydrophilic, unlocked areas on the substrate;
sequencing polynucleotides in the unlocked areas;
rinsing the unlocked areas; and
illuminating the unlocked areas with electromagnetic radiation having a wavelength of about 380 nm to about 1000 nm to deactivate the photo-switchable polymer, thereby returning the previously unlocked areas to a hydrophobic, locked state.

32. The method of claim 31, wherein illuminating comprising illuminating the entire area of the substrate coated with the photo-switchable polymer.

33. The coated substrate of claim 31, wherein the contact angle of the coating in a hydrophobic, locked state is great than 90°, great than 120°, or great than 150°.

34. The coated substrate of any one of claims 31-33, wherein the contact angle of the coated substrate with a photo-switchable polymer in a hydrophilic, unlocked state is less than 90°, less than 30°, or less than 5°.

35. The method of any one of claims 31-34, wherein illuminating one or more areas of a substrate coated with a photo-switchable polymer in a hydrophobic, locked state is done with electromagnetic radiation having a wavelength of about 300 nm to about 380 nm.

36. The method of any one of claims 31-35, wherein illuminating the unlocked areas is done with electromagnetic radiation having a wavelength of about 380 nm to about 780 nm.

37. The method of any one of claims 31-36, wherein sequencing polynucleotides comprises sequencing by synthesis.

38. The method of any one of claims 31-37, wherein rinsing the unlocked areas comprises rinsing with water.


SEQUENCE LISTING (2 sequences)

SEQ ID NO: 1 - DNA, 39 bp, synthetic construct
  source 1..39: mol_type = other DNA
  modified_base 33: mod_base = OTHER; note = Uracil
ttttttttttaatgatacggcgaccaccgagatctacac

SEQ ID NO: 2 - DNA, 34 bp, synthetic construct
  source 1..34: mol_type = other DNA
  modified_base 32: mod_base = OTHER; note = 8-Oxoguanine
ttttttttttcaagcagaagacggcatacgagat